(19) United States
(12) Patent Application Publication
(10) Pub. No.: US 2004/0103449 A1
(43) Pub. Date: May 27, 2004

(54) Identification and use of cytochrome P450 nucleic acid sequences from tobacco

(75) Inventor: Dongmei Xu, Lexington, KY (US)

Correspondence Address: FITCH EVEN TABIN AND FLANNERY, 120 SOUTH LA SALLE STREET, SUITE 1600, CHICAGO, IL, 60603-3406, US

(73) Assignee: U.S. Smokeless Tobacco Company

(21) Appl. No.: 10/293,252

(22) Filed: Nov. 13, 2002

Related U.S. Application Data
(60) Provisional application No. 60/363,684, filed on Mar. 12, 2002.
(60) Provisional application No. 60/347,444, filed on Jan. 11, 2002.
(60) Provisional application No. 60/337,684, filed on Nov. 13, 2001.

(51) Int. Cl. A01H 1/00; C12N 15/82; C12N 9/02; C12N 5/04

(52) U.S. Cl. 800/278; 435/189; 435/320.1; 435/419

(57) ABSTRACT

The present invention relates to P450 enzymes and nucleic acid sequences encoding P450 enzymes in plants, more specifically tobacco, and methods of using those enzymes and nucleic acid sequences to alter plant phenotypes.

The present invention relates to nucleic acid sequences encoding P450 enzymes in tobacco and methods for using those nucleic acid sequences to alter plant phenotypes.

### BACKGROUND

Cytochrome P450s catalyze enzymatic reactions for a diverse range of chemically dissimilar substrates that include the oxidative, peroxidative and reductive metabolism of endogenous and xenobiotic substrates (Danielson, Curr. Drug Metab. 2002, 3:561-597). In plants, P450 enzymes participate in a variety of biochemical pathways including the synthesis of plant products such as phenylpropanoids, alkaloids, terpenoids, lipids, cyanogenic glycosides, and glucosinolates (Chappell, Annu. Rev. Plant Physiol. Plant Mol. Biol. 198, 49:311-343). Cytochrome P450s, also known as P450 hemethiolate proteins, usually act as terminal oxidases in multi-component electron transfer chains, called P450-containing monooxygenase systems. Specific reactions catalyzed include demethylation, hydroxylation, epoxidation, N-oxidation, sulfooxidation, N-, S-, and O-dealkylations, desulfation, deamination, and reduction of azo, nitro, and N-oxide groups.

More than four hundred cytochrome P450 enzymes have been identified in diverse organisms ranging from bacteria, fungi, plants, to animals (Graham-Lorence et al., FASEB J., 1996, 10:206-214.) The B-class of P450 enzymes is found in prokaryotes and fungi, while the E-class is found is found in bacteria, plants, insects, vertebrates, and mammals. At least five subclasses are found within the larger family of E-class cytochrome P450s. All cytochrome P450s use a heme cofactor and share structural attributes. Most cytochrome P450s are 400 to 530 amino acids in length. The secondary structure of the enzyme is about 70% alpha-helical and about 22% beta-sheet. The region around the heme-binding site in the C-terminal part of the protein is conserved among cytochrome P450s. A ten amino acid signature sequence in this hemeiron ligand region has been identified which includes a conserved cysteine involved in binding the heme iron in the fifth coordination site. In eukaryotic cytochrome P450s, a membrane-spanning region is usually found in the first 15-20 amino acids of the protein. Generally, the membrane spanning region consists of approximately 15 hydrophobic residues followed by a positively charged residue (See Graham-Lorence, supra).

The diverse role of tobacco P450 enzymes has been implicated in effecting a variety of plant metabolites such as phenylpropanoids, alkaloids, terpenoids, lipids, cyanogenic glycosides, glucosinolates and a host of other chemical entities. During recent years, it is becoming apparent that some P450 enzymes can impact the composition of metabolites in plants. For example, it has been long desired to improve the flavor and aroma of a burley variety by altering its profile of selected fatty acids through breeding; however, very little is known about mechanisms involved in controlling the levels of these leaf constituents. The down-regulation of P450 enzymes associated with the modification of fatty acids may facilitate accumulation of desired fatty acids that provide more preferred leaf qualities. The function of P450 enzymes and their broadening roles in plant constituents is still being discovered. For instance, a special class of P450 enzymes was found to catalyze the breakdown of fatty acid into volatile C6- and C9-aldehydes and -alcohols that are major contributors of “fresh green” odor of fruits and vegetables (Noordermeer et al, Chembiochem 2001, 2: 494-504). The level of other novel targeted P450 enzymes may be altered to enhance the qualities of leaf constituents by modifying lipid composition and related break down metabolites in tobacco leaf. Still other reports have shown that P450s enzymes are capable of producing cyanogenic glucoside from gluucosinolate compounds that may have utility in improving disease resistance (Bak et al, Plant Physiol 2000, 123: 1437-1448).

In other instances, P450 enzymes have been suggested to be involved in alkaloid biosynthesis. Nornicotine is a minor alkaloid found in tobacco. It is supposedly produced by the P450 demethylation of nicotine, and is then readily acylated and nitrosated at the N position thereby producing a series of N-acylnonicotines and N-nitrosonornicotines. N-demethylation catalyzed by a tobacco demethylase is thought to be a primary source of nornicotine biosyntheses in tobacco. Tobacco nicotine demethylase is believed to be microsomal and possibly a P-450 dependent enzyme. Thus far a soluble nicotine demethylase enzyme has not been successfully purified, nor have the genes involved been isolated.

The activity of P450 enzymes is genetically controlled and also strongly influenced by environment factors. For example, the demethylation of nicotine to form nornicotine in tobacco is thought to increase substantially when the plants reach a mature stage. Furthermore, it is thought that the demethylase gene contains a transposable element that can inhibit translation of RNA when present. However, the transposable element can be easily excised when the plant is stressed by environmental factors or artificially by treatment with hormones or other components, thus resulting in protein production and subsequent nornicotine production. This explains why non-nornicotine tobacco lines (non-convertor lines) can convert to nornicotine producing lines (convertor lines) when placed in tissue culture or when seed is continually inbred through the practice of repeatedly saving seed and then using that saved seed for further seed production. For example, ethylene is thought to indirectly stimulate nornicotine production by accelerating senescence.

The large multiplicity of P450 forms, their differing structure and function have made research on P450 very difficult. The cloning of P450s has been hampered at least in part because these membrane-localized proteins are typically present in low abundance and often unstable to purification. Hence, a need exists for the identification of P450 enzymes in plants and the nucleic acid sequences associated with those P450 enzymes.

### SUMMARY

The present invention is directed to plant P450 enzymes and to plant P450 enzymes having enzymatic activity. The present invention is also directed to P450 enzymes in plants whose expression is induced by ethylene and/or plant senescence. The present invention is further directed to nucleic acid sequences in plants that encode P450 enzymes having activities such as oxigenase, demethylase, and other and the use of those sequences to reduce or silence the expression of these enzymes. The invention also relates to P450 enzymes found in plants expressing higher nornicotine levels as opposed to P450 enzymes found in plants exhibiting lower nornicotine levels.

In one aspect, the invention is directed to nucleic acid sequences as set forth in SEQ. ID. Nos. 13, 15, 17,19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151. These nucleic acid sequences may then be utilized to reduce, or more preferably, silence or knock out cytochrome P450 enzymes transcription or translation in plants. Reduction or elimination of P450 transcription or translation and subsequent reduction in protein concentration and/or enzymatic activity is accomplished by introducing nucleic acid sequences into the plant using techniques commonly available to one having ordinary skill in the art. Methods for using the nucleic acid sequences taught herein to lower or eliminate P450 enzyme expression using RNA, DNA or protein strategies thereby altering the plant metabolite composition, include without limitation antisense technology, RNA interference (RNAi), GenoPlasty (ValiGen Co.), antibodies, ribozymes, cosuppression/transgene silencing, viral expression systems, mutagenesis, chimeraplasty, and the like. In another aspect, the reduction or elimination of P450 enzymatic activity in plants and more preferably in tobacco may be accomplished transiently using RNA viral vector silencing systems. Resulting transformed or infected plants are assessed for phenotypic changes including, but not limited to, analysis of endogenous P450 RNA transcripts, analysis of P450 expressed peptides, and alterations on of plant metabolite concentrations using techniques commonly available to one having ordinary skill in the art.

In a second aspect, the present invention is also directed to generation of trangenic plant lines such as tobaccos that have altered P450 enzyme activity levels whereby such transgenic tobacco lines produce altered levels of metabolites. In accordance with the invention, these transgenic lines include nucleic acid sequences that are effective for reducing or silencing the expression of enzymes that play a role in the demethylation, hydroxylation, epoxidation, N-oxidation, sulfooxidation, N-, S-, and O-dealkylations, desulfation, and deamination reactions as well as reactions involving the reduction of azo, nitro, and N-oxide groups. Such nucleic acid sequences include SEQ. ID. Nos. 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

In this aspect of the invention, the nucleic acids are operably linked to a promoter that is functional in the plant to provide a transformation vector. The plant or plant cells are transformed with the transformer vector and transformed cells are selected. The selected cells are then regenerated into a plant. In accordance with the invention, the nucleic acid molecule may be in an antisense orientation, a sense orientation, or RNA interference orientation. The nucleic and may be expressed as a double standard RNA molecule. The double standard RNA molecule may be about 15 to 25 nucleotides in length.

In a further aspect of the invention, plant cultivars including nucleic acids of the present invention in a down regulation capacity will have altered metabolite profiles relative to control plants.

In a third aspect, the present invention is directed to the screening of plants, more preferably tobacco, that contain genes that have substantial nucleic acid identity to the taught nucleic acid sequence. The use of the invention is advantageous to identify and select plants that contain a nucleic acid sequence with exact or substantial identity where such plants are part of a breeding program for traditional or transgenic varieties, a mutagenesis program, or naturally occurring diverse plant populations. The screening of plants for substantial nucleic acid identity may be accomplished by evaluating plant nucleic acid materials using a nucleic acid probe in conjunction with nucleic acid detection protocols including, but not limited to, nucleic acid hybridization and PCR detection and the like. The nucleic acid probe may comprise nucleic acid sequence or fragment thereof corresponding to SEQ ID 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

In a fourth aspect, the present invention is directed to the identification of plant genes, more preferably tobacco plant genes, encoding proteins that share substantial amino acid identity corresponding to the taught nucleic acid sequence. The identification of a nucleic acid sequence with substantial identity may be accomplished by screening plant cDNA libraries using a nucleic acid probe in conjunction with nucleic acid detection protocols including, but not limited to, nucleic acid hybridization, PCR analysis, and the like. The nucleic acid probe may be comprised of nucleic acid sequence or fragment thereof corresponding to SEQ ID 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151. Alternatively, cDNA expression libraries that express peptides may be screened using antibodies directed to part or all of the taught amino acid sequence taught herein. Such amino acid sequences include SEQ ID 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, or 152.

### BRIEF DESCRIPTION OF DRAWINGS

FIG. 1 shows a procedure used for cloning of cytochrome P450 cDNA fragements by PCR. SEQ. ID. Nos. 1-12 are shown.

FIG. 2 shows nucleic acid SEQ. ID. No.:13 and amino acid SEQ. ID. No.:14.

FIG. 3 shows nucleic acid SEQ. ID. No.:15 and amino acid SEQ. ID. No.:16.

FIG. 4 shows nucleic acid SEQ. ID. No.:17 and amino acid SEQ. ID. No.:18.

FIG. 5 shows nucleic acid SEQ. ID. No.:19 and amino acid SEQ. ID. No.:20.

FIG. 6 shows nucleic acid SEQ. ID. No.:21 and amino acid SEQ. ID. No.:22.

FIG. 7 shows nucleic acid SEQ. ID. No.:23 and amino acid SEQ. ID. No.:24.

FIG. 8 shows nucleic acid SEQ. ID. No.:25 and amino acid SEQ. ID. No.:26.

FIG. 9 shows nucleic acid SEQ. ID. No.:27 and amino acid SEQ. ID. No.:28.

FIG. 10 shows nucleic acid SEQ. ID. No.:29 and amino acid SEQ. ID. No.:30.

FIG. 11 shows nucleic acid SEQ. ID. No.:31 and amino acid SEQ. ID. No.:32.

FIG. 12 shows nucleic acid SEQ. ID. No.:33 and amino acid SEQ. ID. No.:34.

FIG. 13 shows nucleic acid SEQ. ID. No.:35 and amino acid SEQ. ID. No.:36.

FIG. 14 shows nucleic acid SEQ. ID. No.:37 and amino acid SEQ. ID. No.:38.

FIG. 15 shows nucleic acid SEQ. ID. No.:39 and amino acid SEQ. ID. No.:40.

FIG. 16 shows nucleic acid SEQ. ID. No.:41 and amino acid SEQ. ID. No.:42.

FIG. 17 shows nucleic acid SEQ. ID. No.:43 and amino acid SEQ. ID. No.:44.

FIG. 18 shows nucleic acid SEQ. ID. No.:45 and amino acid SEQ. ID. No.:46.

FIG. 19 shows nucleic acid SEQ. ID. No.:47 and amino acid SEQ. ID. No.:48.

FIG. 20 shows nucleic acid SEQ. ID. No.:49 and amino acid SEQ. ID. No.:50.

FIG. 21 shows nucleic acid SEQ. ID. No.:51 and amino acid SEQ. ID. No.:52.

FIG. 22 shows nucleic acid SEQ. ID. No.:53 and amino acid SEQ. ID. No.:54.

FIG. 23 shows nucleic acid SEQ. ID. No.:55 and amino acid SEQ. ID. No.:56.

FIG. 24 shows nucleic acid SEQ. ID. No.:57 and amino acid SEQ. ID. No.:58.

FIG. 25 shows nucleic acid SEQ. ID. No.:59 and amino acid SEQ. ID. No.:60.

FIG. 26 shows nucleic acid SEQ. ID. No.:61 and amino acid SEQ. ID. No.:62.

FIG. 27 shows nucleic acid SEQ. ID. No.:63 and amino acid SEQ. ID. No.:64.

FIG. 28 shows nucleic acid SEQ. ID. No.:65 and amino acid SEQ. ID. No.:66.

FIG. 29 shows nucleic acid SEQ. ID. No.:67 and amino acid SEQ. ID. No.:68.

FIG. 30 shows nucleic acid SEQ. ID. No.:69 and amino acid SEQ. ID. No.:70.

FIG. 31 shows nucleic acid SEQ. ID. No.:71 and amino acid SEQ. ID. No.:72.

FIG. 32 shows nucleic acid SEQ. ID. No.:73 and amino acid SEQ. ID. No.:74.

FIG. 33 shows nucleic acid SEQ. ID. No.:75 and amino acid SEQ. ID. No.:76.

FIG. 34 shows nucleic acid SEQ. ID. No.:77 and amino acid SEQ. ID. No.:78.

FIG. 35 shows nucleic acid SEQ. ID. No.:79 and amino acid SEQ. ID. No.:80.

FIG. 36 shows nucleic acid SEQ. ID. No.:81 and amino acid SEQ. ID. No.:82.

FIG. 37 shows nucleic acid SEQ. ID. No.:83 and amino acid SEQ. ID. No.:84.

FIG. 38 shows nucleic acid SEQ. ID. No.:85 and amino acid SEQ. ID. No.:86.

FIG. 39 shows nucleic acid SEQ. ID. No.:87 and amino acid SEQ. ID. No.:88.

FIG. 40 shows nucleic acid SEQ. ID. No.:89 and amino acid SEQ. ID. No.:90.

FIG. 41 shows nucleic acid SEQ. ID. No.:91 and amino acid SEQ. ID. No.:92.

FIG. 42 shows nucleic acid SEQ. ID. No.:93 and amino acid SEQ. ID. No.:94.

FIG. 43 shows nucleic acid SEQ. ID. No.:95 and amino acid SEQ. ID. No.:96.

FIG. 44 shows nucleic acid SEQ. ID. No.:97 and amino acid SEQ. ID. No.:98.

FIG. 45 shows nucleic acid SEQ. ID. No.:99 and amino acid SEQ. ID. No.:100.

FIG. 46 shows nucleic acid SEQ. ID. No.:101 and amino acid SEQ. ID. No.:102.

FIG. 47 shows nucleic acid SEQ. ID. No.:103 and amino acid SEQ. ID. No.:104.

FIG. 48 shows nucleic acid SEQ. ID. No.:105 and amino acid SEQ. ID. No.:106.

FIG. 49 shows nucleic acid SEQ. ID. No.:107 and amino acid SEQ. ID. No.:108.

FIG. 50 shows nucleic acid SEQ. ID. No.:109 and amino acid SEQ. ID. No.:110.

FIG. 51 shows nucleic acid SEQ. ID. No.:111 and amino acid SEQ. ID. No.:112.

FIG. 52 shows nucleic acid SEQ. ID. No.:113 and amino acid SEQ. ID. No.:114.

FIG. 53 shows nucleic acid SEQ. ID. No.:115 and amino acid SEQ. ID. No.:116.

FIG. 54 shows nucleic acid SEQ. ID. No.:117 and amino acid SEQ. ID. No.:118.

FIG. 55 shows nucleic acid SEQ. ID. No.:119 and amino acid SEQ. ID. No.:120.

FIG. 56 shows nucleic acid SEQ. ID. No.:121 and amino acid SEQ. ID. No.:122.

FIG. 57 shows nucleic acid SEQ. ID. No.:123 and amino acid SEQ. ID. No.:124.

FIG. 58 shows nucleic acid SEQ. ID. No.:125 and amino acid SEQ. ID. No.:126.

FIG. 59 shows nucleic acid SEQ. ID. No.:127 and amino acid SEQ. ID. No.:128.

FIG. 60 shows nucleic acid SEQ. ID. No.:129 and amino acid SEQ. ID. No.:130.

FIG. 61 shows nucleic acid SEQ. ID. No.:131 and amino acid SEQ. ID. No.:132.

FIG. 62 shows nucleic acid SEQ. ID. No.:133 and amino acid SEQ. ID. No.:134.

FIG. 63 shows nucleic acid SEQ. ID. No.:135 and amino acid SEQ. ID. No.:136.

FIG. 64 shows nucleic acid SEQ. ID. No.:137 and amino acid SEQ. ID. No.:138.

FIG. 65 shows nucleic acid SEQ. ID. No.:139 and amino acid SEQ. ID. No.:140.

FIG. 66 shows nucleic acid SEQ. ID. No.:141 and amino acid SEQ. ID. No.:142.

FIG. 67 shows nucleic acid SEQ. ID. No.:143 and amino acid SEQ. ID. No.:144.

FIG. 68 shows nucleic acid SEQ. ID. No.:145 and amino acid SEQ. ID. No.:146.

FIG. 69 shows nucleic acid SEQ. ID. No.:147 and amino acid SEQ. ID. No.:148.

FIG. 70 shows nucleic acid SEQ. ID. No.:149 and amino acid SEQ. ID. No.:150.

FIG. 71 shows nucleic acid SEQ. ID. No.:151 and amino acid SEQ. ID. No.:152.

### DETAILED DESCRIPTION

Definitions

Unless defined otherwise, all technical and scientific terms used herein have meanings as commonly understood by one of ordinary skill in the art to which this invention belongs. Singleton et al. (1994) Dictionary of Microbiology and Molecular Biology, second edition, John Wiley and Sons (New York) provides one of skill with a general dictionary of many of the terms used in this invention. All patents and publications referred to herein are incorporated by reference herein. For purposes of the present invention, the following terms are defined below.

The term “cytochrome P450, P450 and P-450” are used herein interchangeably.

The term “nucleic acid” refers to a deoxyribonucleotide or ribonucleotide polymer in either single- or double-stranded form, or sense or anti-sense, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof. The terms “operably linked”, “in operable combination”, and “in operable order” refer to functional linkage between a nucleic acid expression control sequence (such as a promoter, signal sequence, matrix attachment regions, or array of transcription factor binding sites and the like) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, expresses said nucleic acid or expresses a peptide, heterologous peptide, or protein encoded by a heterologous nucleic acid. Recombinant cells can express genes or gene fragments in either the sense or antisense form that are not found within the native (non-recombinant) form of the cell. Recombinant cells can also express genes that are found in the native form of the cell, but wherein the genes are modified and re-introduced into the cell by artificial means.

A “structural gene” is that portion of a gene comprising a DNA segment encoding a protein, polypeptide or a portion thereof, and excluding the 5′ sequence which drives the initiation of transcription. The structural gene may alternatively encode a nontranslatable product. The structural gene may be one which is normally found in the cell or one which is not normally found in the cell or cellular location wherein it is introduced, in which case it is termed a “heterologous gene”. A heterologous gene may be derived in whole or in part from any source known to the art, including a bacterial genome or episome, eukaryotic, nuclear or plasmid DNA, cDNA, viral DNA or chemically synthesized DNA. A structural gene may contain one or more modifications which could effect biological activity or its characteristics, the biological activity or the chemical structure of the expression product, the rate of expression or the manner of expression control. Such modifications include, but are not limited to, mutations, insertions, deletions and substitutions of one or more nucleotides. The structural gene may constitute an uninterrupted coding sequence or it may include one or more introns, bounded by the appropriate splice junctions. The structural gene may be translatable or non-translatable, including in an anti-sense orientation, RNAi configuration or the like. The structural gene may be a composite of segments derived from a plurality of sources and from a plurality of gene sequences (naturally occurring or synthetic, where synthetic refers to DNA that is chemically synthesized).

“Derived from” is used to mean taken, obtained, received, traced, replicated or descended from a source (chemical and/or biological). A derivative may be produced by chemical or biological manipulation (including, but not limited to, substitution, addition, insertion, deletion, extraction, isolation, mutation and replication) of the original source.

“Chemically synthesized”, as related to a sequence of DNA, means that portions of the component nucleotides were assembled in vitro. Manual chemical synthesis of DNA may be accomplished using well established procedures (Caruthers, Methodology of DNA and RNA Sequencing, (1983), Weissman (ed.), Praeger Publishers, New York, Chapter 1); automated chemical synthesis can be performed using one of a number of commercially available machines standard in the art.

Two polynucleotides or polypeptides are said to be “identical” if the sequence of nucleotides or amino acid residues in the two sequences is the same when aligned for maximum correspondence. Optimal alignment of sequences for comparison may be conducted by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman Proc. Natl. Acad. Sci. (U.S.A.) 85: 2444 (1988), by computerized implementations of these computerized algorithms (e.g., GAP, BESTFIT, FASTA, and TFASTA in the Wisconsin Genetics Software Package, Genetics Computer Group, 575 Science Dr., Madison, Wis.), or by inspection.

The NCBI Basic Local Alignment Search Tool (BLAST) (Altschul et al., 1990) is available from several sources, including the National Center for Biological Information (NCBI, Bethesda, Md.) and on the Internet, for use in connection with the sequence analysis programs blastp, blastn, blastx, tblastn and tblastx. It can be accessed at htp://www.ncbi.nlm.nih.gov/BLAST/. A description of how to determine sequence identity using this program is available at http://www.ncbi.nlm.nih.gov/BLAST/blast help.html.

The terms “substantial identity” or “substantial sequence identity” as applied to nucleic acid sequences and as used herein denote a characteristic of a polynucleotide sequence, wherein the polynucleotide comprises a sequence that has at least 80 percent sequence identity, at least 80 to 99 percent sequence identity being desired, preferably at least 90 to 99 percent sequence identity, more preferably at least 95 to 99 percent sequence identity, and most preferably at least 98 to 99 as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence.

Another indication that nucleotide sequences are substantially identical is if two molecules hybridize to each other under stringent conditions. Stringent conditions are sequence-dependent and will be different in different circumstances. Generally, stringent conditions are selected to be about 5° C. to about 20° C., usually about 10° C. to about 15° C., lower than the thermal melting point (Tm) for the specific sequence at a defined ionic strength and pH. The Tm is the temperature (under defined ionic strength and pH) at which 50% of the target sequence hybridizes to a matched probe. Typically, stringent conditions will be those in which the salt concentration is about 0.02 molar at pH 7 and the temperature is at least about 60° C. For instance in a standard Southern hybridization procedure, stringent conditions will include an initial wash in 6×SSC at 42° C. followed by one or more additional washes in 0.2×SSC at a temperature of at least about 55° C., typically about 60° C. and often about 65° C.

Nucleotide sequences are also substantially identical for purposes of this invention when the polypeptides and/or proteins which they encode are substantially identical. Thus, where one nucleic acid sequence encodes essentially the same polypeptide as a second nucleic acid sequence, the two nucleic acid sequences are substantially identical, even if they would not hybridize under stringent conditions due to degeneracy permitted by the genetic code (see, Darnell et al. (1990) Molecular Cell Biology, Second Edition Scientific American Books W. H. Freeman and Company New York for an explanation of codon degeneracy and the genetic code). Protein purity or homogeneity can be indicated by a number of means well known in the art, such as polyacrylamide gel electrophoresis of a protein sample, followed by visualization upon staining. For certain purposes high resolution may be needed and HPLC or a similar means for purification may be utilized.

As used herein, the term “vector” is used in reference to nucleic acid molecules that transfer DNA segment(s) into a cell. A vector may act to replicate DNA and may reproduce independently in a host cell. Vectors may be of fungal, bacterial, viral, animal or plant origin. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eucaryotic cells are known to utilize promoters, enhancers, and termination and polyadenylation signals. Viral vectors will often require those elements consistent with those used in prokaryotic and eukaryotic systems.

For the purpose of regenerating complete genetically engineered plants or plant cell tissues, a nucleic acid may be inserted into plant cells, for example, by any technique such as in vivo inoculation or by any of the known in vitro tissue culture techniques to produce transformed plant cells that can be regenerated into complete plants. Thus, for example, the insertion into plant cells may be by in vitro inoculation by pathogenic or non-pathogenic A. tumefaciens. Other such tissue culture techniques may also be employed.

Transcriptional control signals in eukaryotes comprise “promoters” and may comprise “enhancer” elements. Promoters and enhancers consist of short arrays of DNA sequences that interact specifically with cellular proteins involved in transcription (Maniatis, T. et al., Science 236:1237 (1987)). Promoter and enhancer elements have been isolated from a variety of eukaryotic sources including genes in yeast, insect and mammalian cells, plants and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, S. D. et al., Trends Biochem. Sci., 11:287 (1986) and Maniatis, T. et al., supra (1987)).

“Plant tissue” includes differentiated and undifferentiated tissues of plants, including, but not limited to, roots, shoots, leaves, pollen, seeds, tumor tissue and various forms of cells in culture, such as single cells, protoplasts, embryos and callus tissue. The plant tissue may be in planta or in organ, tissue or cell culture.

“Plant cell” as used herein includes plant cells in planta and plant cells and protoplasts in culture.

“cDNA” or “complementary DNA” generally refers to a single stranded DNA molecule with a nucleotide sequence that is complementary to an RNA molecule. cDNA is formed by the action of the enzyme reverse transcriptase on an RNA template.

Strategies for Obtaining Nucleic Acid Sequences

In accordance with the present invention, RNA was extracted from tobacco tissue of converter and non-converter tobacco lines. This extracted RNA was then used to create cDNA. Nucleic acid sequences of the present invention were then generated using two different strategies.

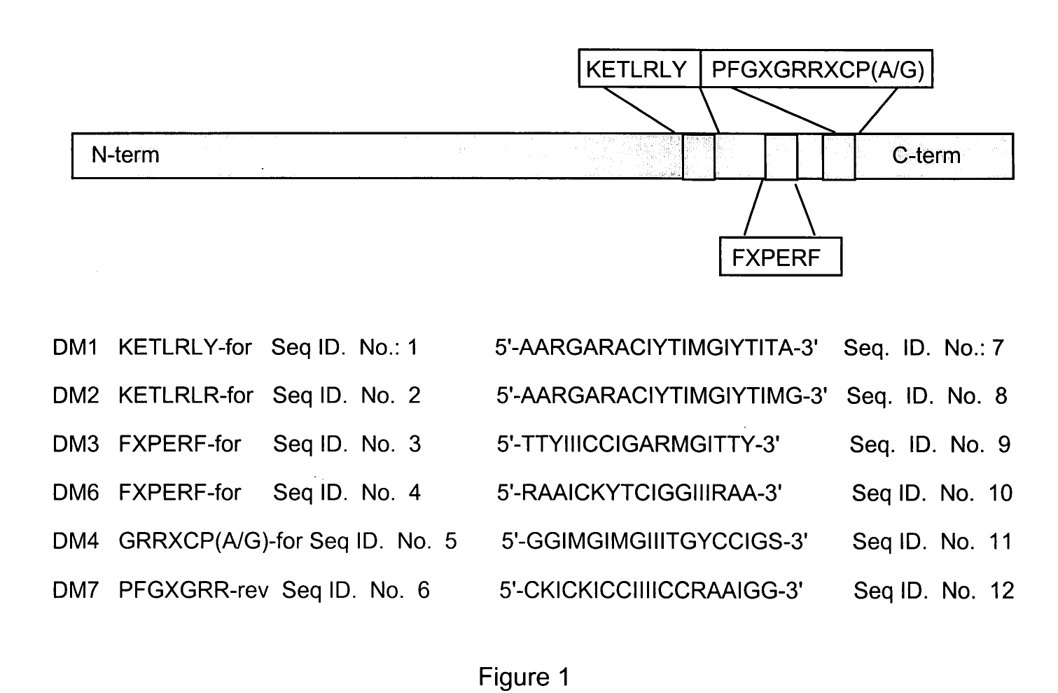

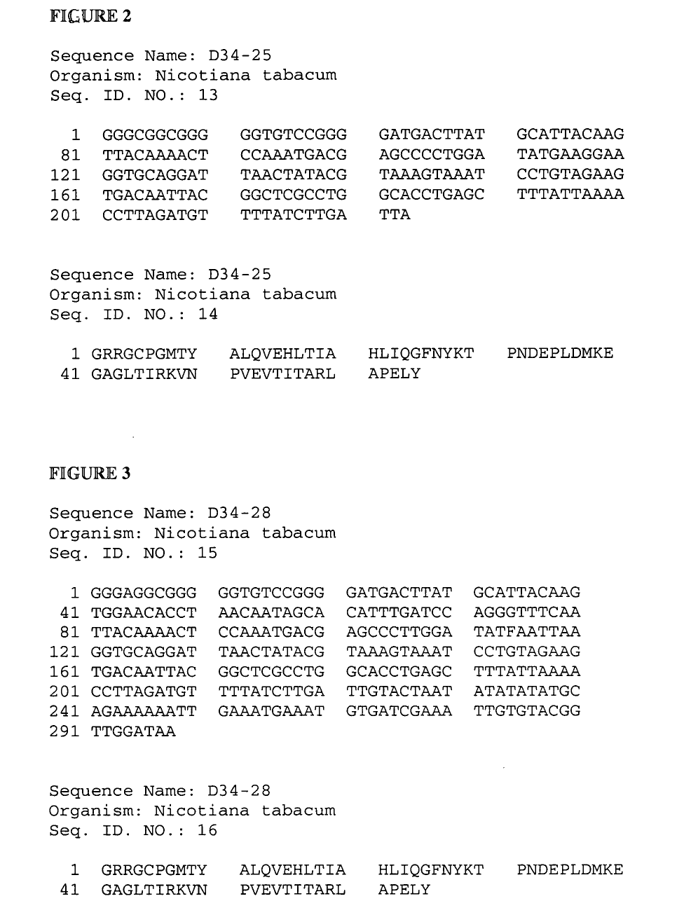

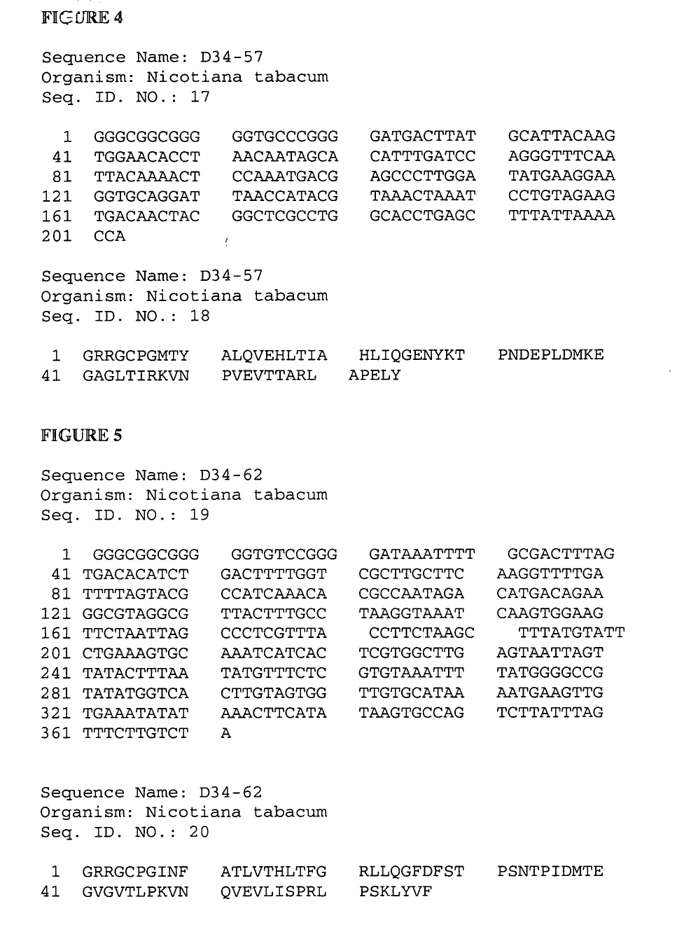

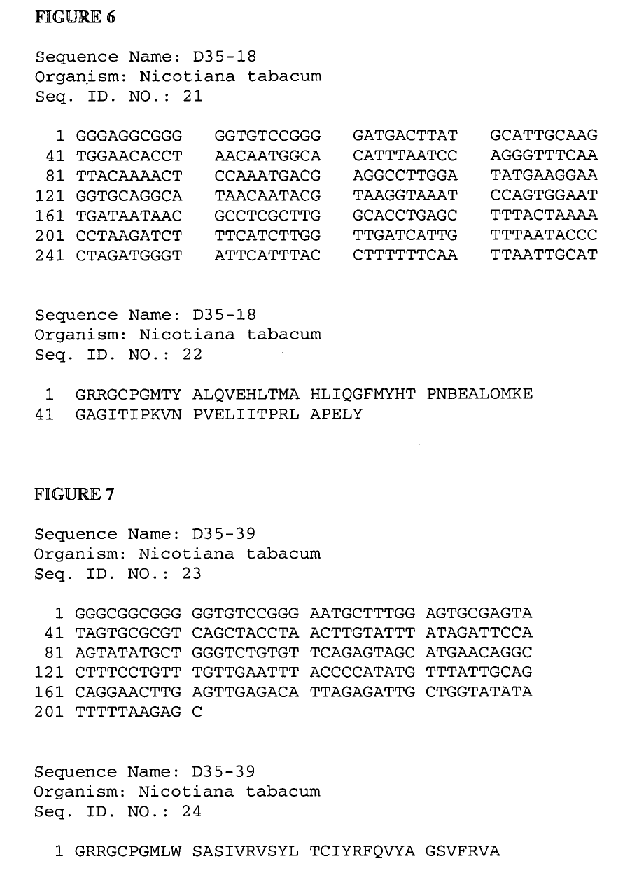

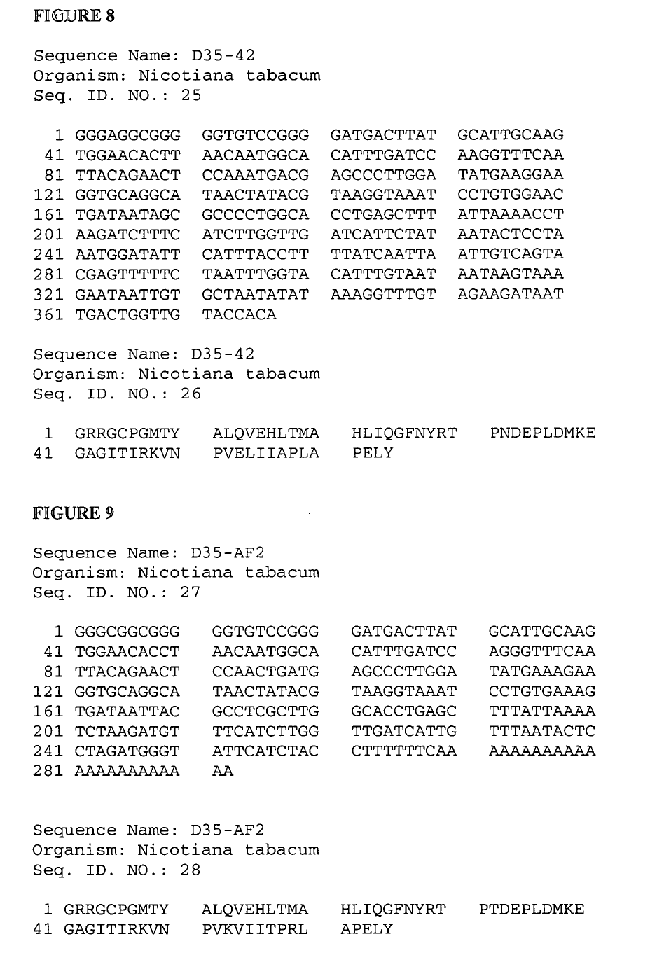

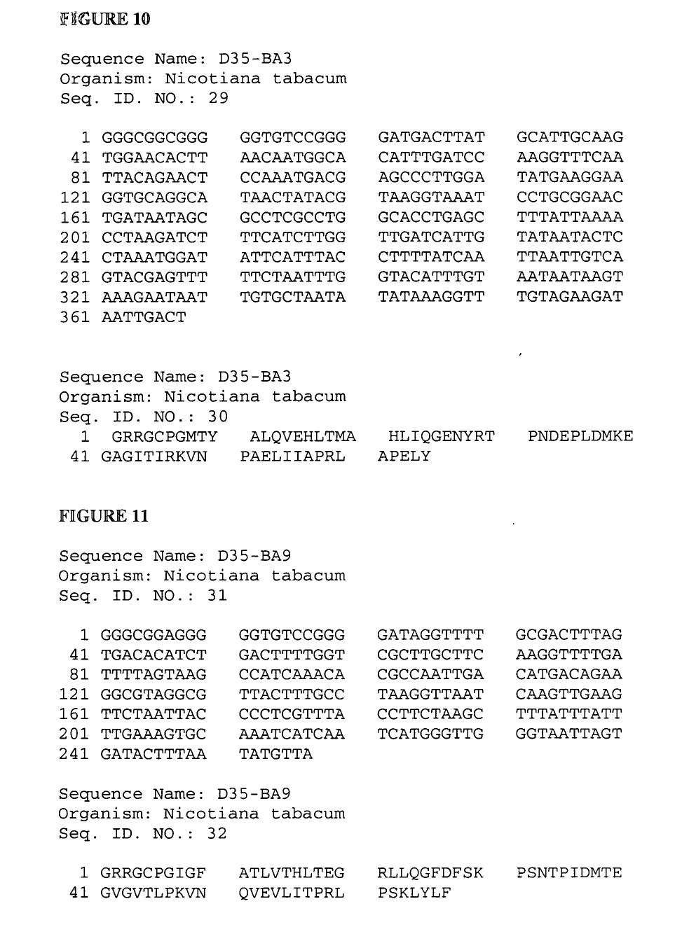

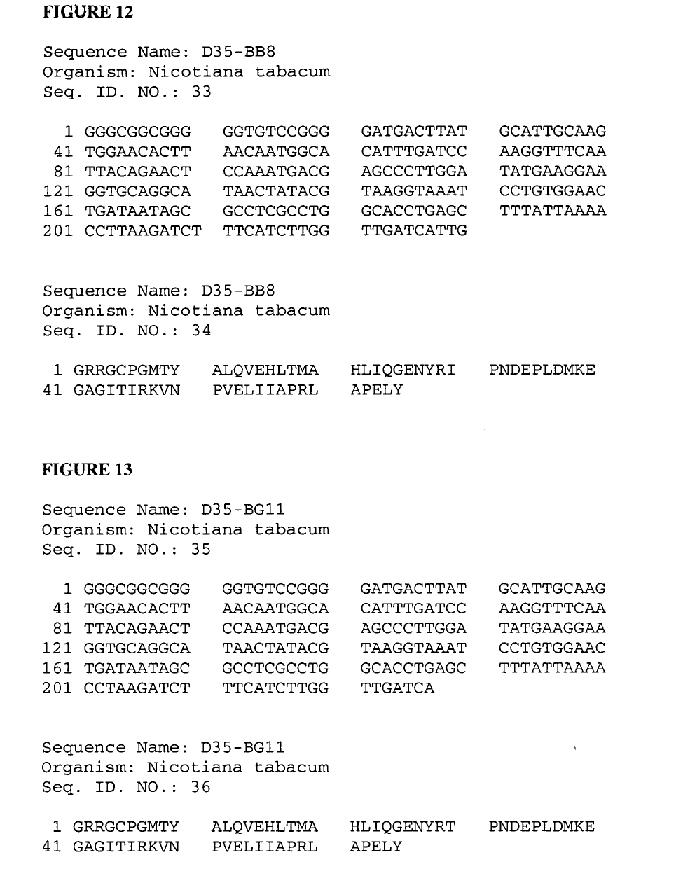

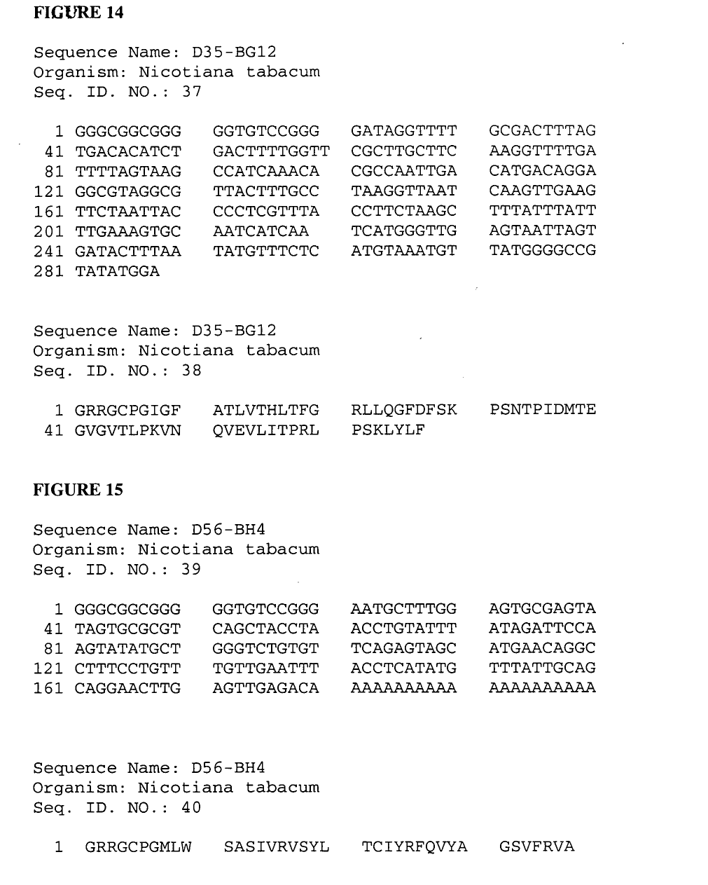

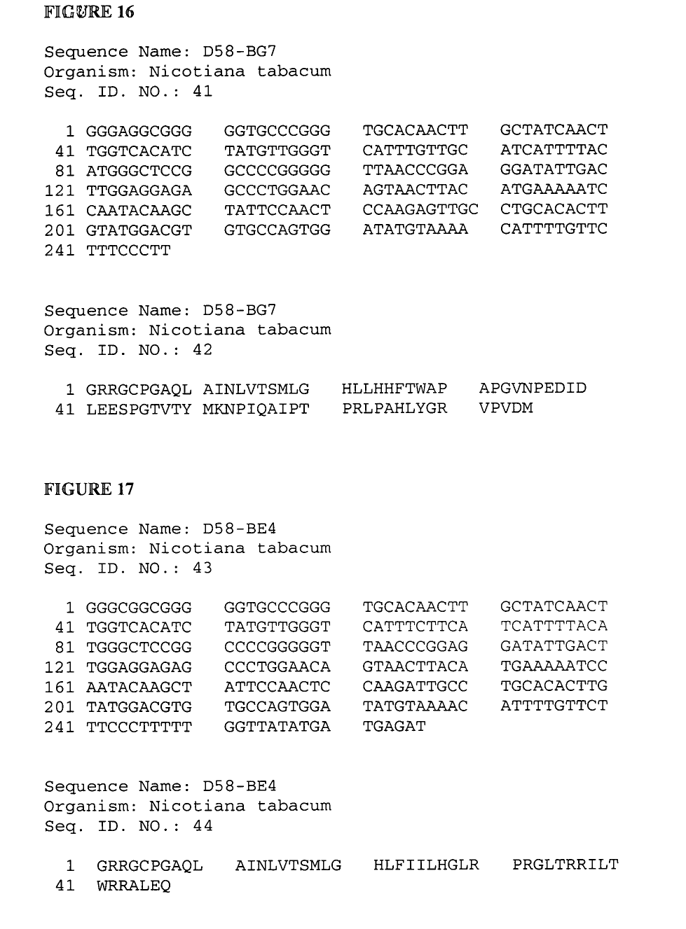

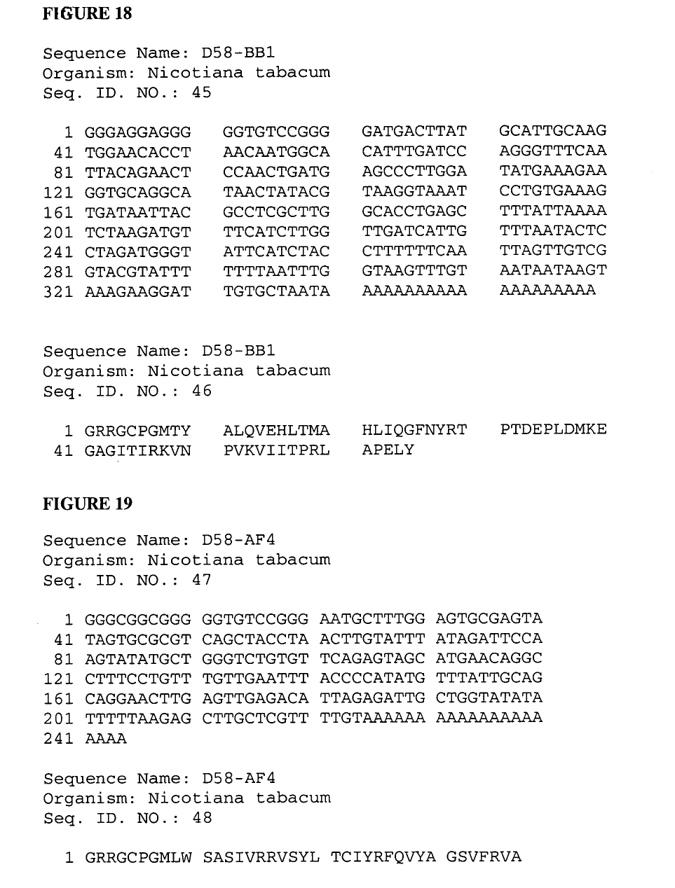

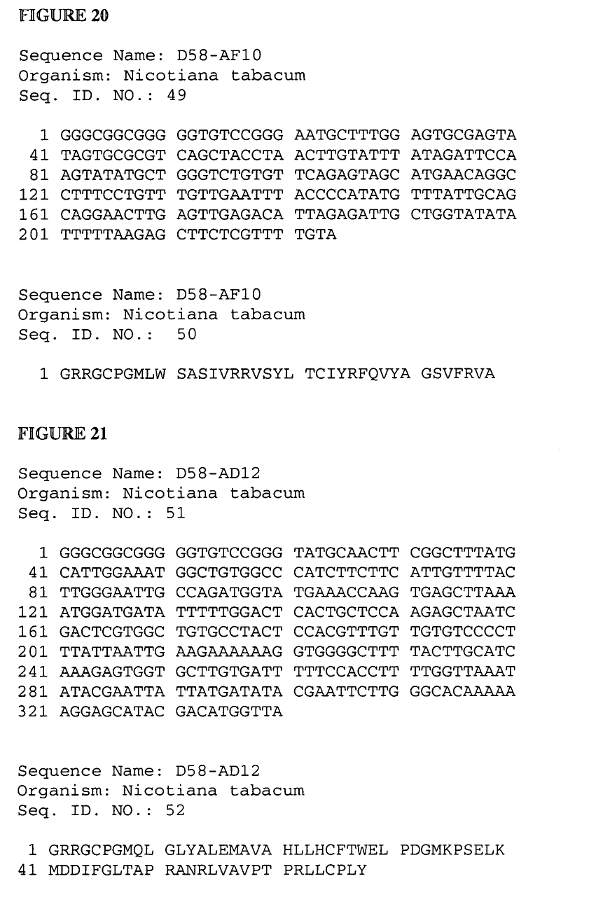

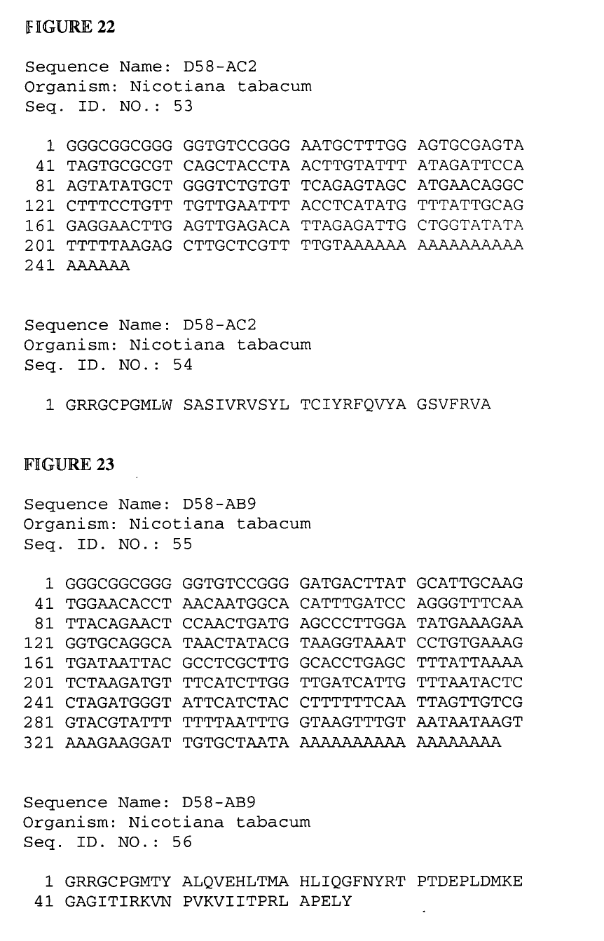

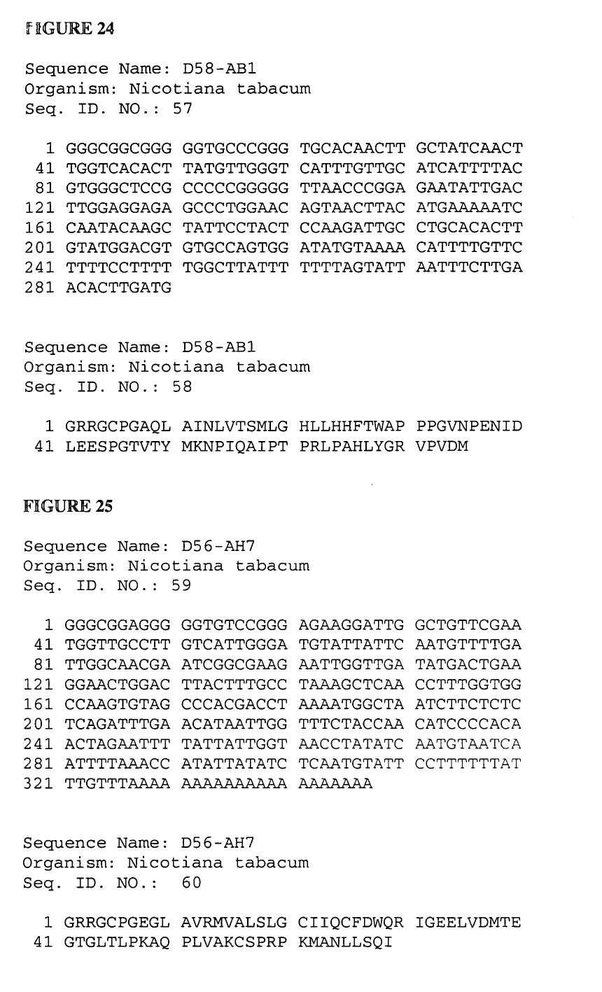

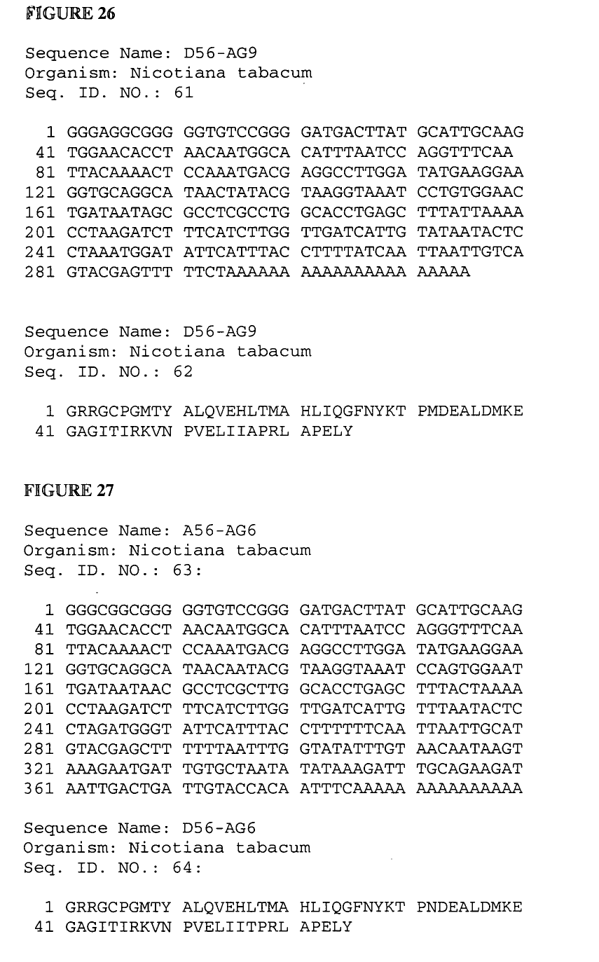

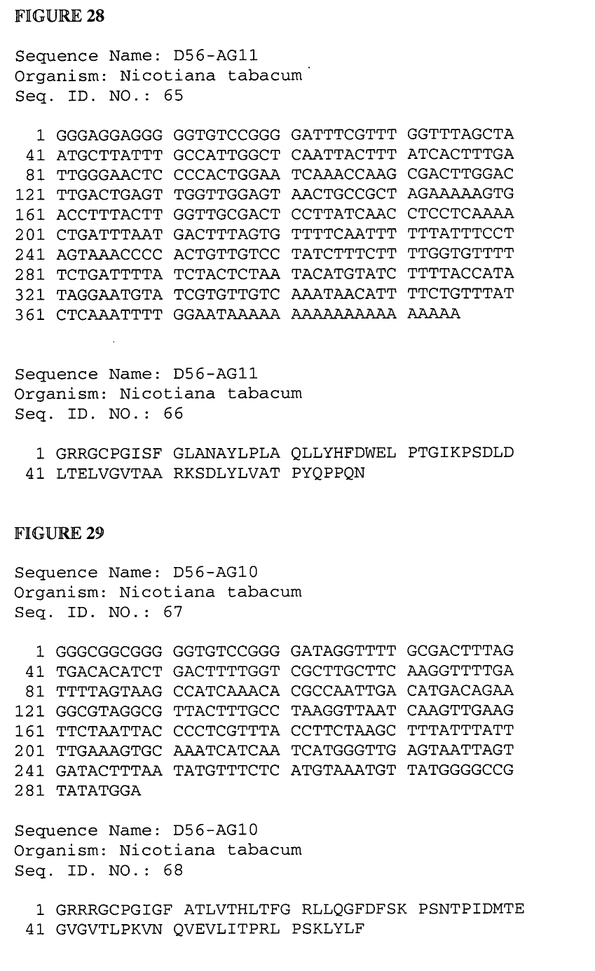

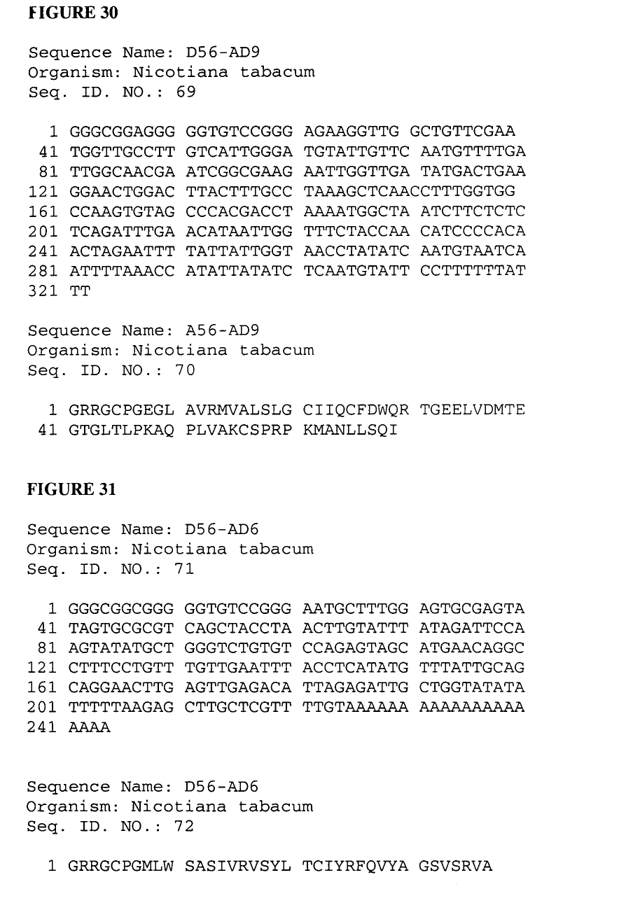

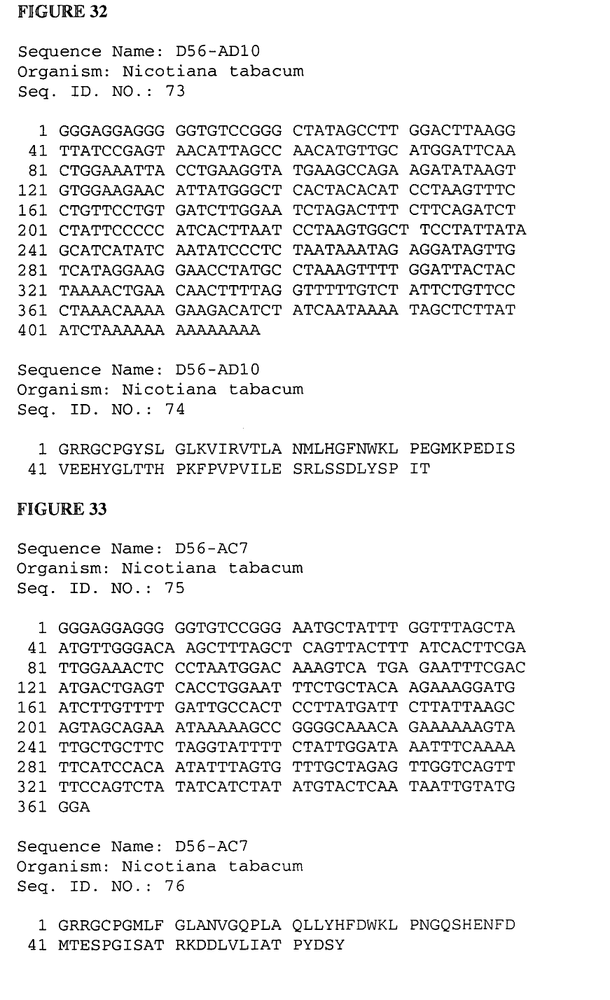

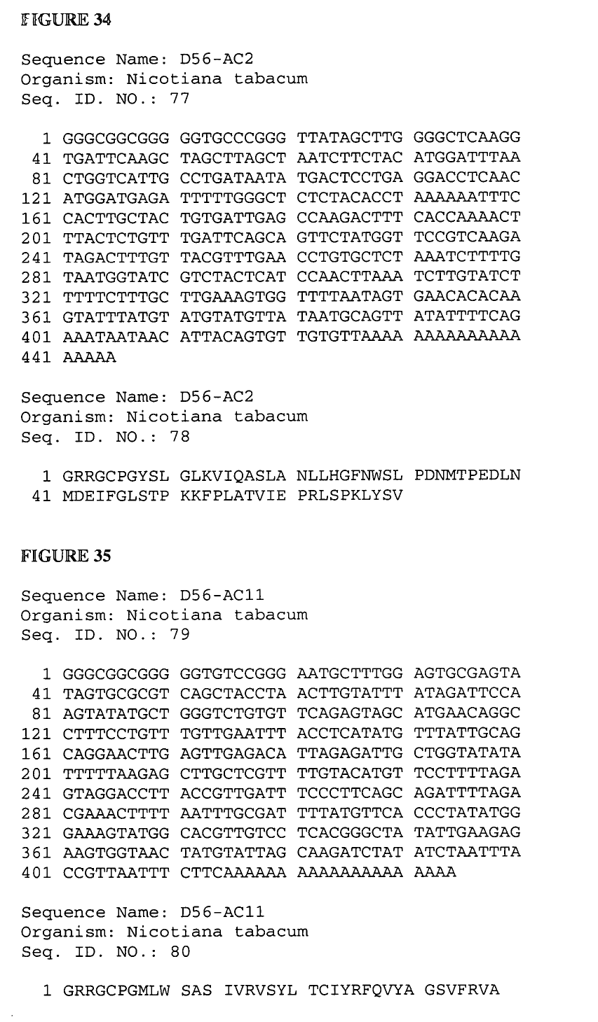

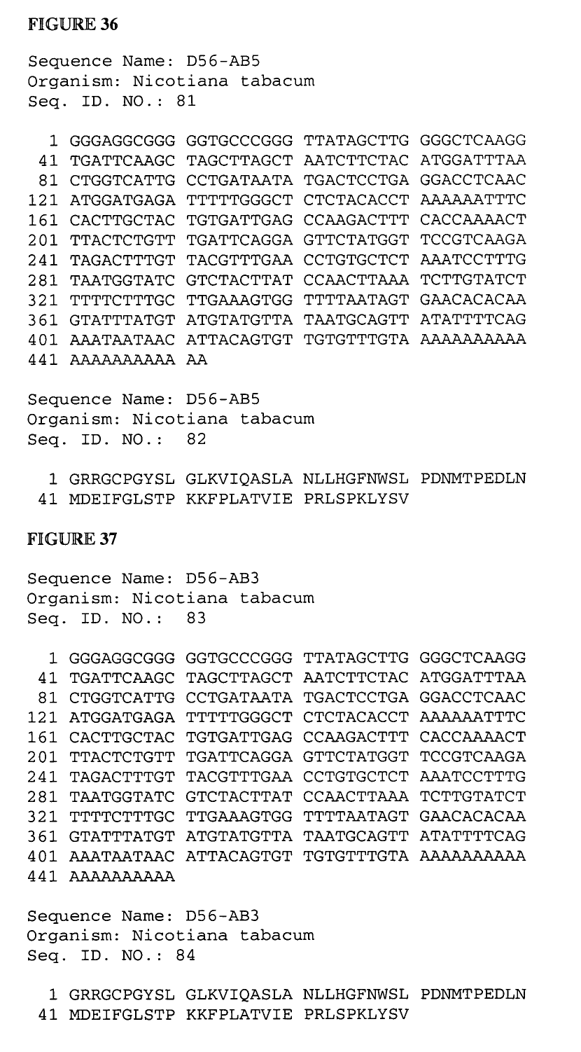

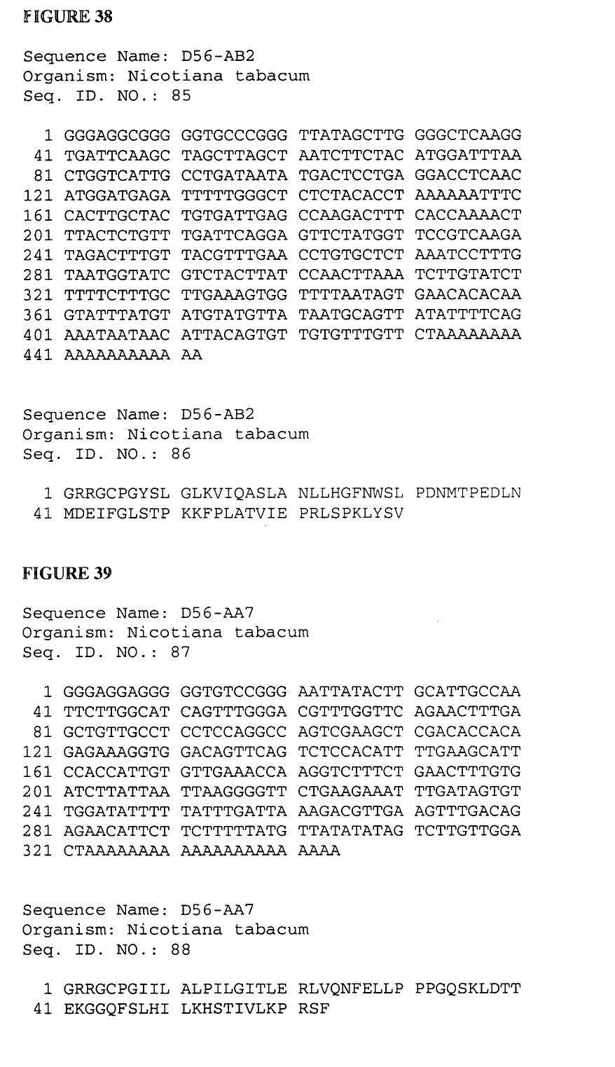

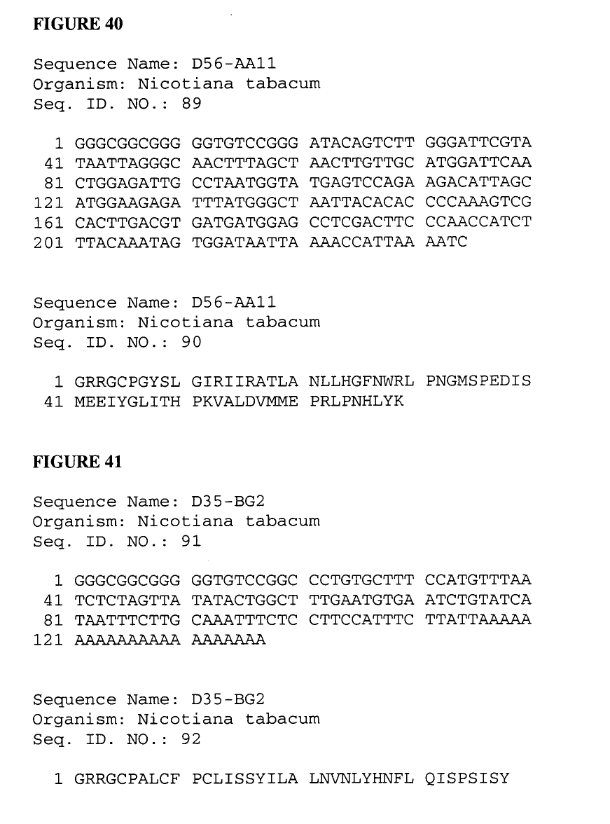

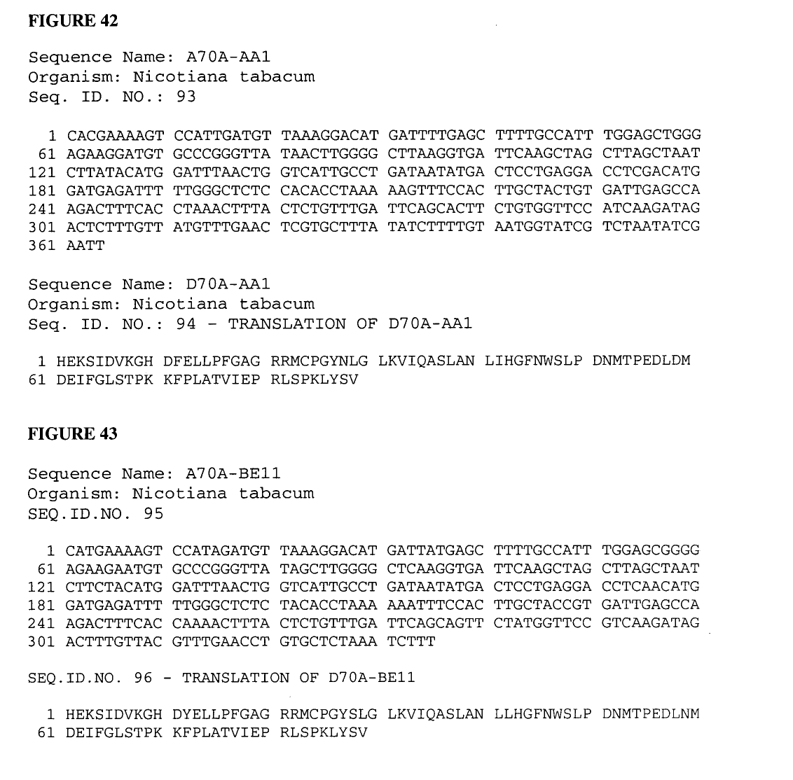

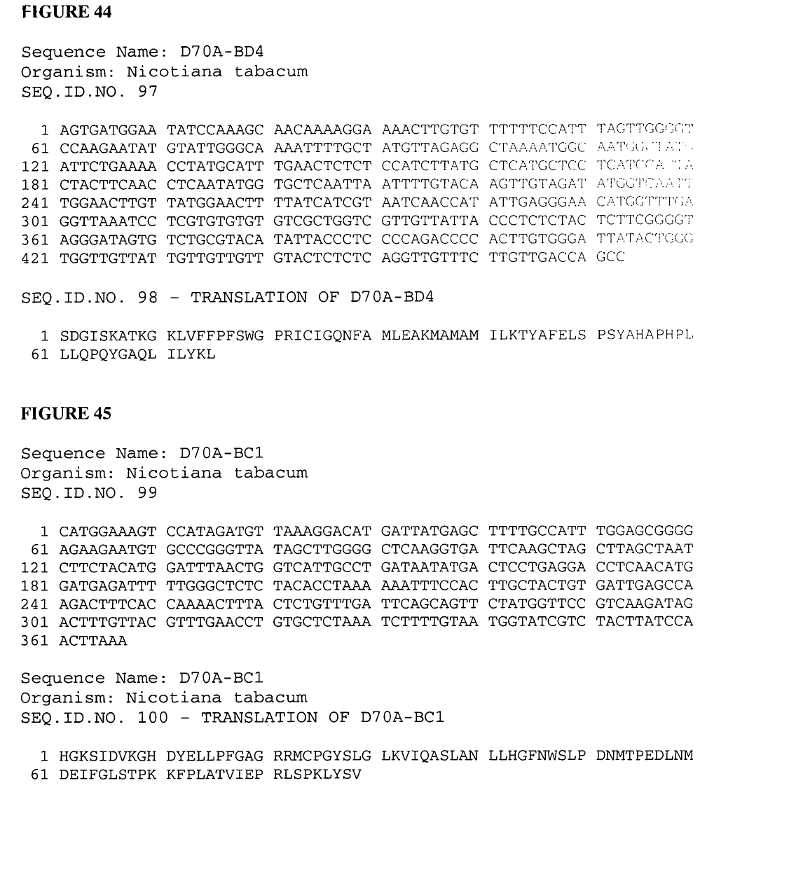

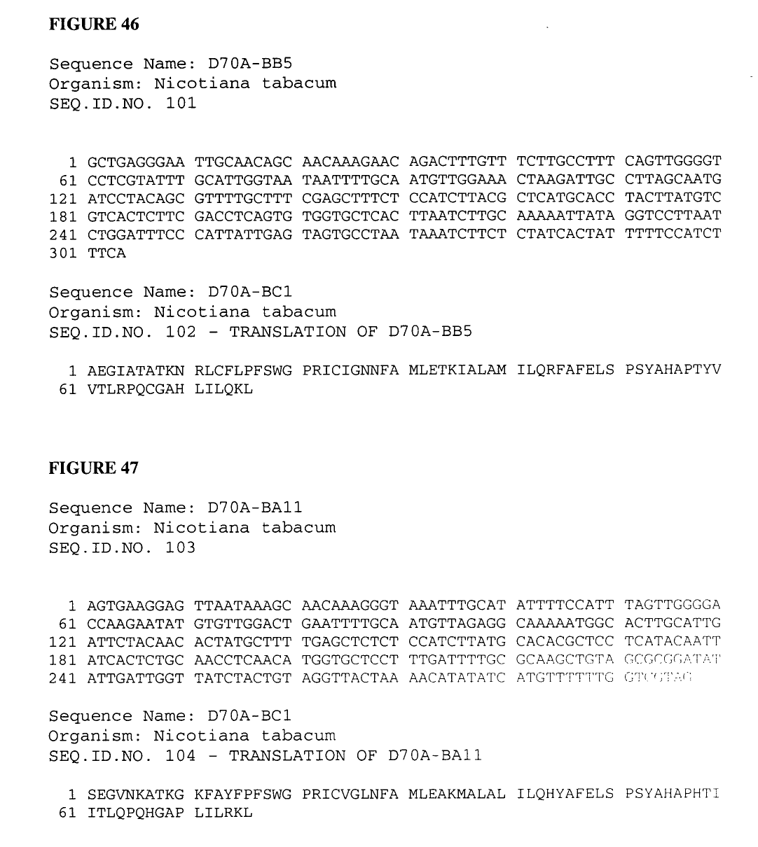

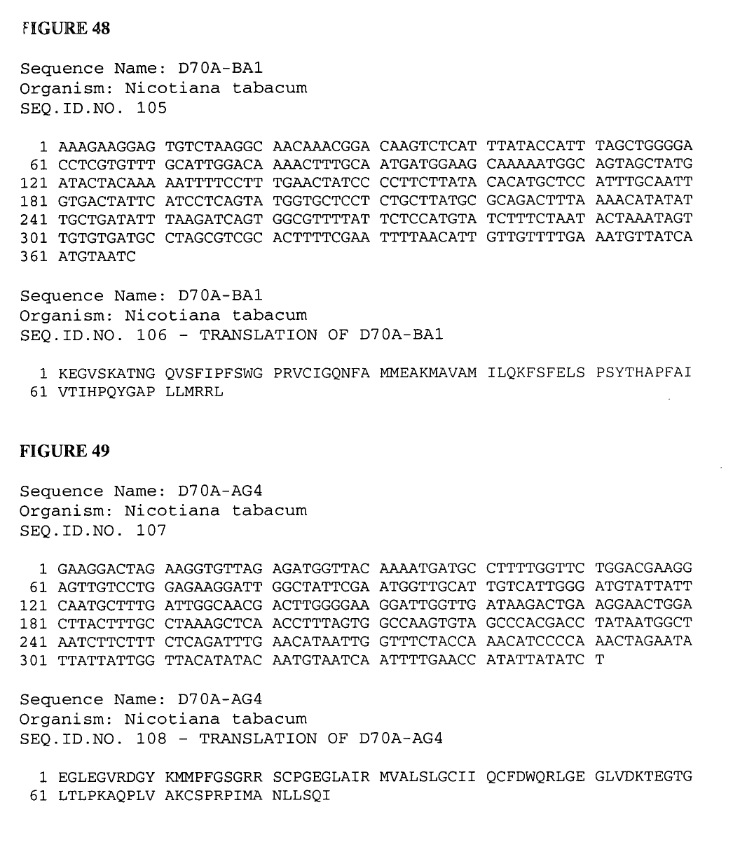

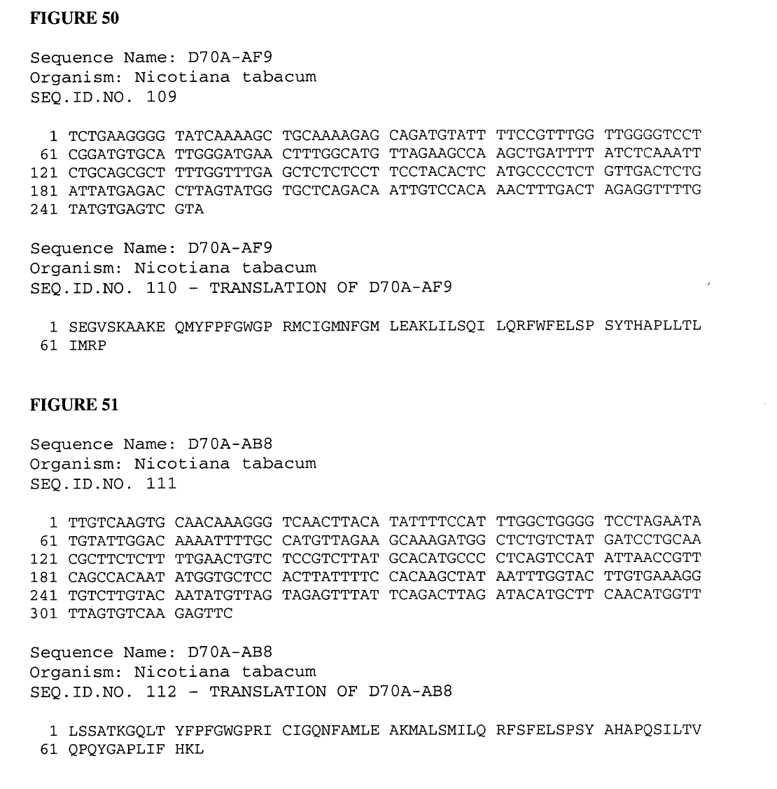

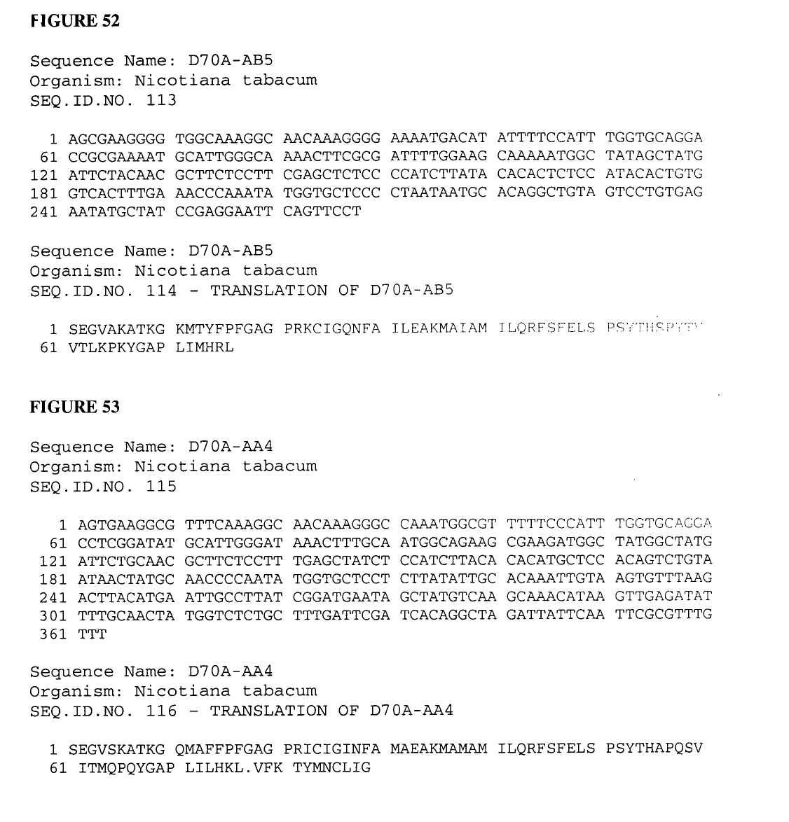

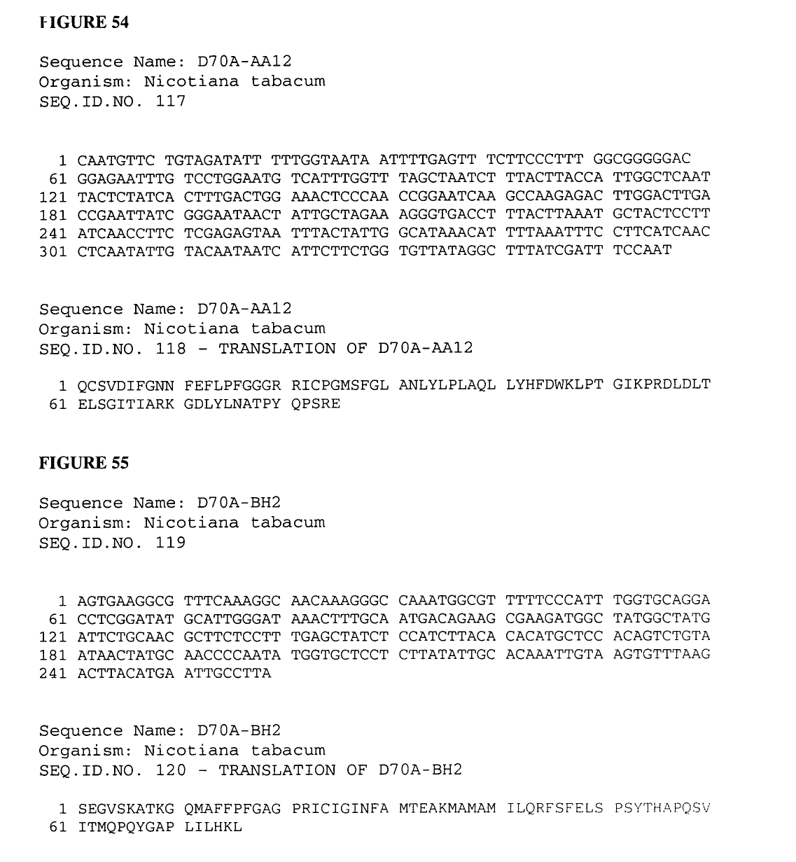

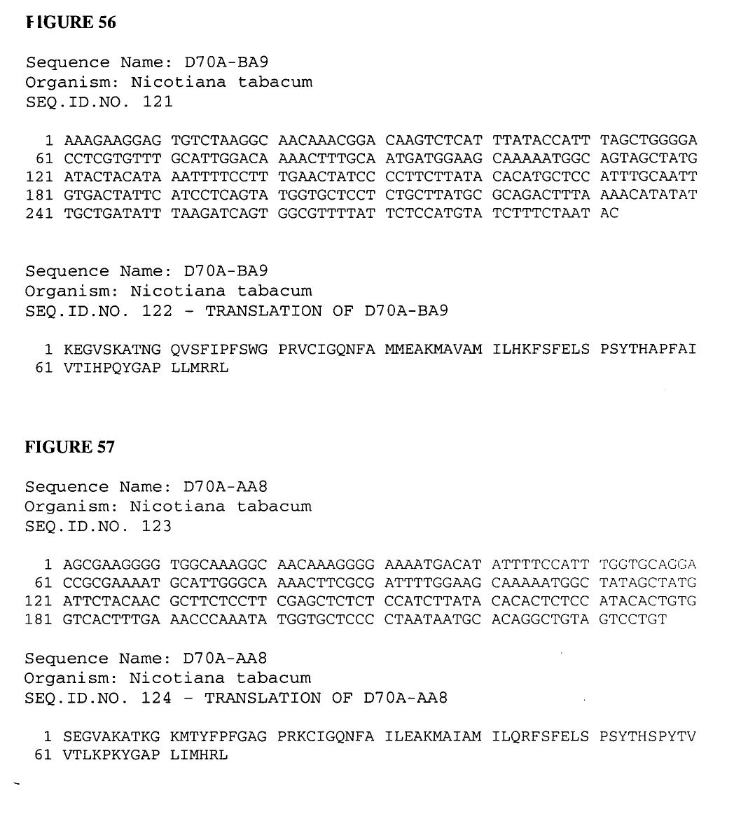

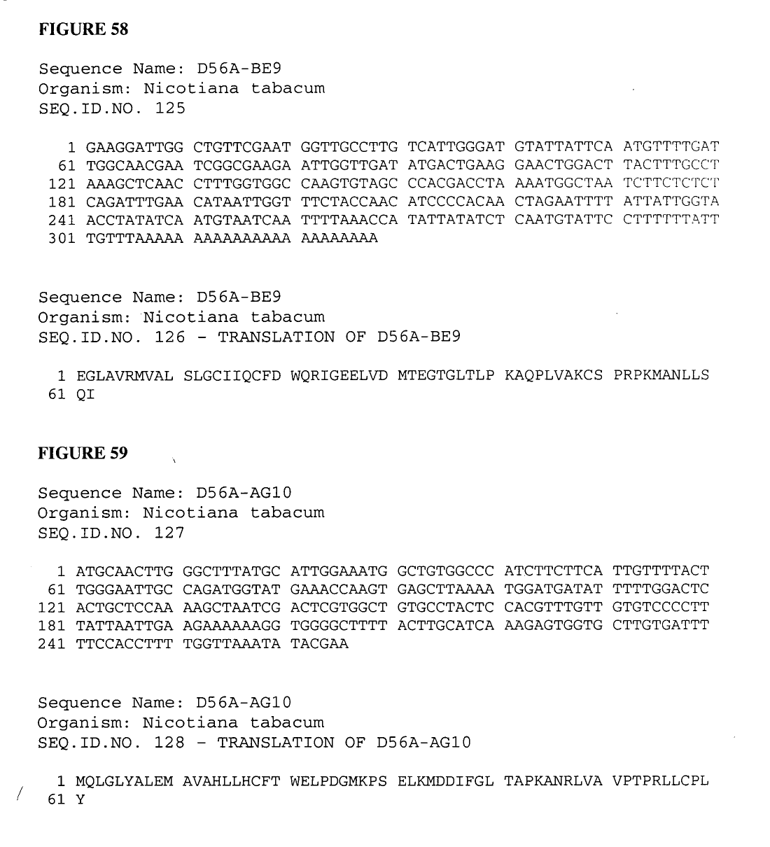

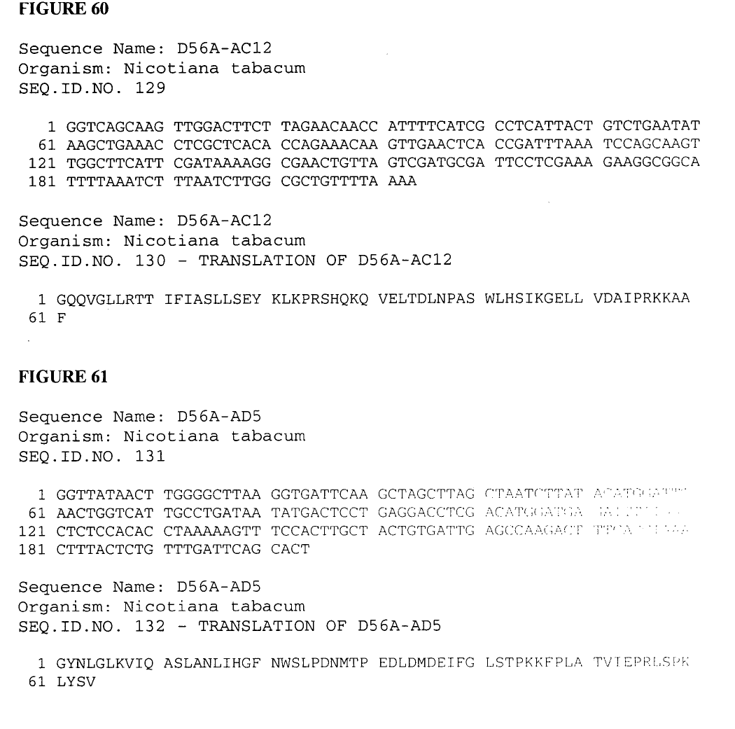

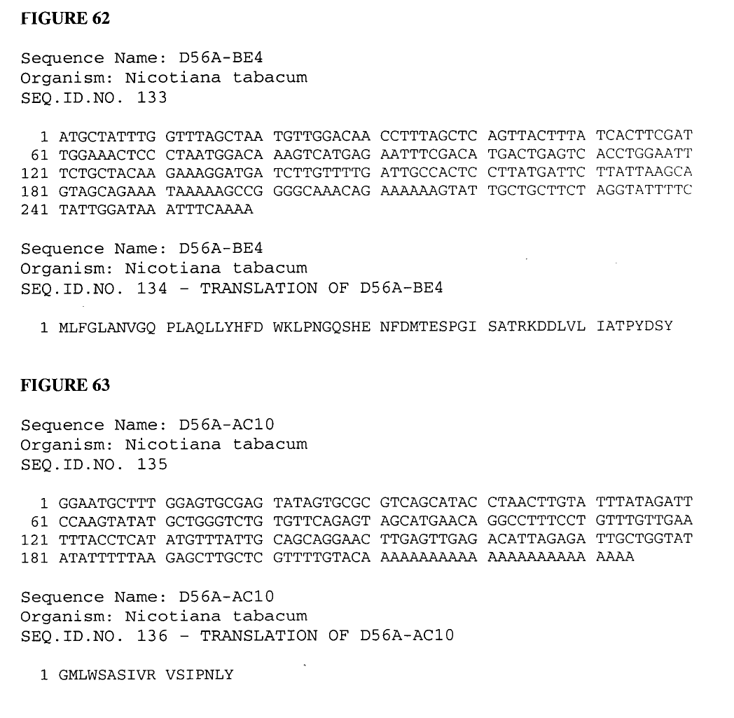

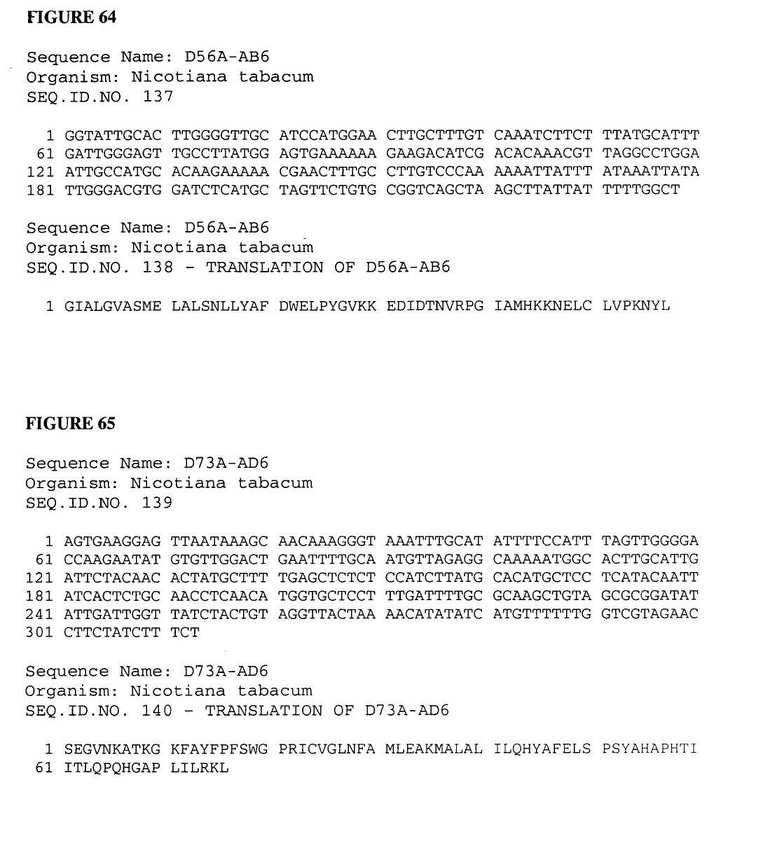

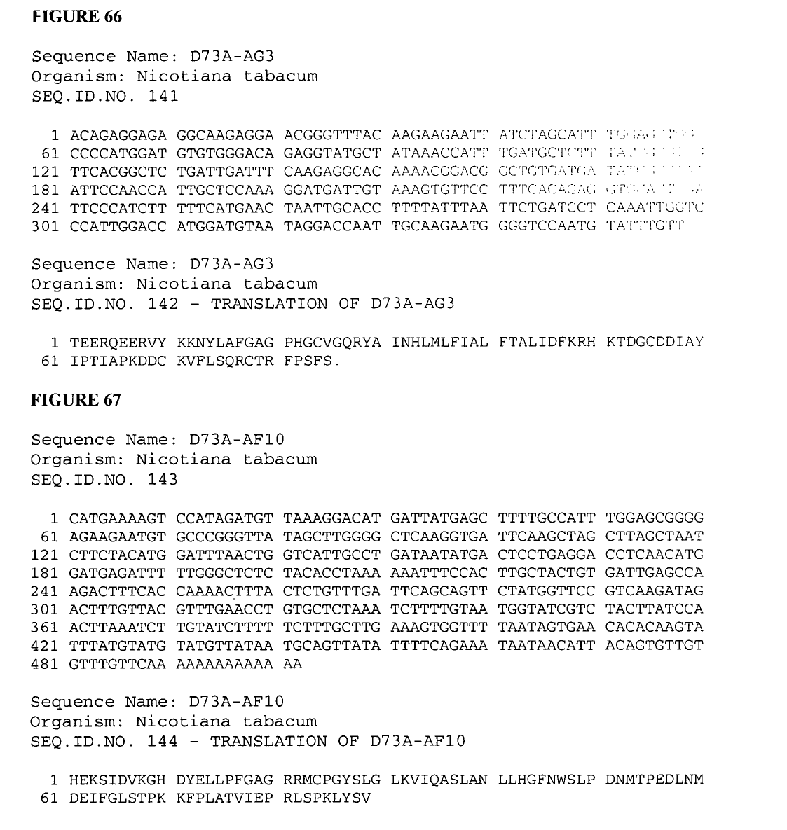

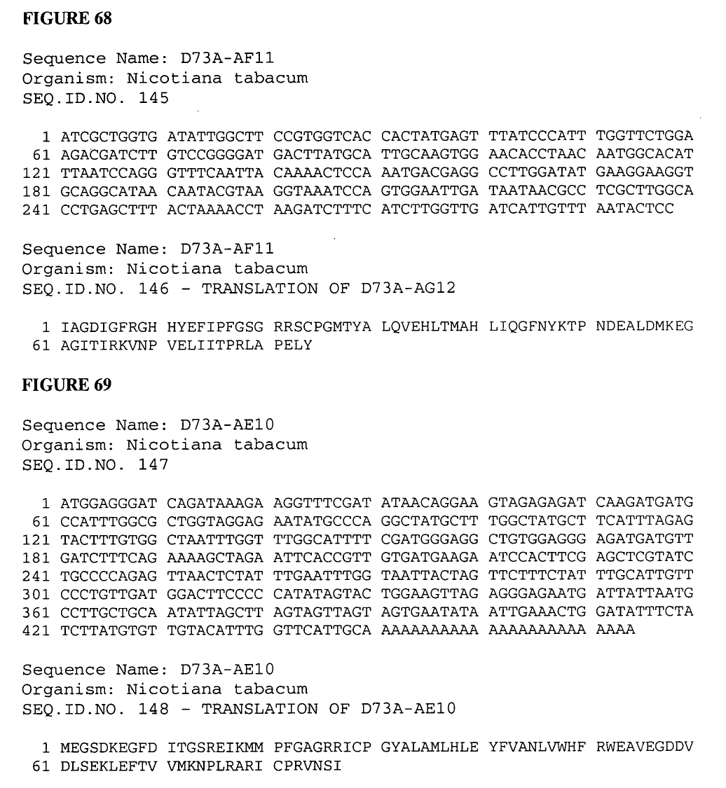

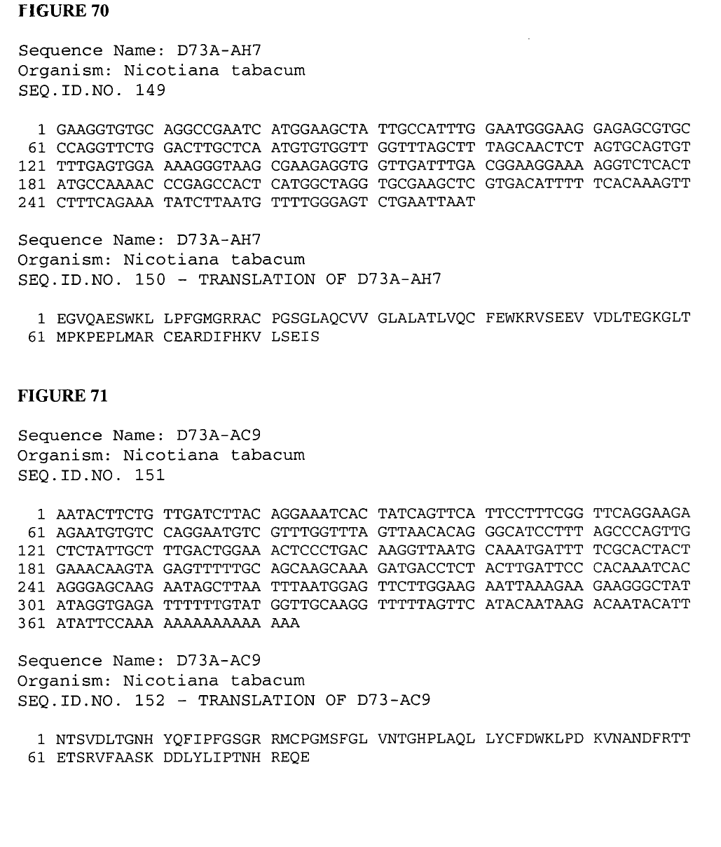

In the first strategy, the cDNA was used to create cytochrome P450 specific PCR populations using degenerate primers. Examples of specific degenerate primers are set forth in FIG. 1. Sequence fragments from plasmids containing appropriate size inserts were further analyzed. These size inserts typically ranged from about 300 to about 800 nucleotides depending on which primers were used.

In a second strategy, the cDNA was used to create subtraction libraries. As in the first strategy, sequence fragments from plasmids containing appropriate size inserts were further analyzed.

Plant Cell Material: Tobacco plant lines known to produce high levels of nornicotine (converter) and plant lines having undetectable levels of nornicotine by gas chromatography/mass spectroscopy may be used as starting materials. In one aspect of the invention, a burley line, variety 4407, lines 58-33 (converter) and 58-25 (nonconverter) may be used. There were no obvious phenotypic differences between these converter lines except for nornicotine levels. Burley converter line 78379 may also be utilized.

cDNA Isolation: Leaves were removed from plants and treated with ethylene to activate cytochrome P450 activity. Total RNA was extracted using techniques known in the art. cDNA fragments were generated using PCR (RT-PCR) with the primers as described in FIG. 1.

Gene Fragment Identification: Two methods were used for gene fragment identification as follows. (1) The conserved region of P450 type enzymes was used as a template for degenerate primers (FIG. 1). Using degenerate primers, P450 specific nucleic acids were amplified by PCR. Bands indicative of P450-like enzymes were identified by DNA sequencing. PCR fragments were characterized using BLAST search, alignment or other tools to identify appropriate candidates.

(2) cDNA was used to generate subtraction libraries using techniques known to the skilled artisan. Appropriate fragments were ligated to a vector, such as a pGEM vector, and characterized by sequencing and comparative RT-PCR.

Characterization of cDNA: Sequence information from identified fragments was used to develop PCR primers. These primers are used to conduct quantitative RT-PCR from the RNA's of converter and non-converter ethylene treated plant tissue. Only appropriate sized DNA bands (300-800 bp) from converter lines or bands with higher density denoting higher expression in converter lines were used for further characterization. Large scale Southern analysis were conducted to examine the differential expression for all clones obtained. In this aspect of the invention, these large scale Southern assays were conducted using labeled total cDNA's from different tissues as a probe to hybridize with cloned DNA fragments in order to screen all cloned inserts.

Functional Analysis of DNA Fragments

Nucleic acid sequences identified as described above are examined by using virus induced gene silencing technology (VIGS, Baulcombe, Current Opinions in Plant Biology, 1999, 2:109-113).

In another aspect of the invention, interfering RNA technology (RNAi) and related double stranded RNA technologies is used to further characterize gene fragments of the present invention. The following references which describe this technology are incorporated by reference herein, Smith et al., Nature, 2000, 407:319-320; Fire et al., Nature, 1998, 391:306-311; Waterhouse et al., PNAS, 1998, 95:13959-13964; Stalberg et al., Plant Molecular Biology, 1993, 23:671-683; Baulcombe, Current Opinions in Plant Biology, 1999, 2:109-113; and Brigneti et al., EMBO Journal, 1998, 17(22):6739-6746.

P450 Fragments: P450 fragments were identified from populations. Distinct P450 clusters were identified.

Two subtraction libraries were made using 58-33 (converter) as tester and 58-25 (non-converter) as driver. Fragments from clones of the first library were identified as encoding P450 enzymes based on PCR reactions using P450 degenerate primers (DM4 in FIG. 1).

Development of Transegenic Tobacco Cell Lines

In this aspect of the invention, appropriate cDNA fragments as identified above were transformed into tobacco plants to generate knockouts or reduce expression of P-450 like enzyme activities. Plants may be transformed using RNAi techniques (Chuang and Meyerwoitz, 2000, PNAS 97: 495-4990; Vaucheret et al 2001, J Cell Sci 114: 3083-3091), antisense techniques, or a variety of other methods described known to the skilled artisan.

Several techniques exist for introducing foreign genetic material into plant cells, and for obtaining plants that stably maintain and express the introduced gene. Such techniques include acceleration of genetic material coated onto microparticles directly into cells (U.S. Pat. No. 4,945,050 to Cornell and U.S. Pat. No. 5,141,131 to DowElanco). Plants may be transformed using Agrobacterium technology, see U.S. Pat. No. 5,177,010 to University of Toledo, U.S. Pat. No. 5,104,310 to Texas A&M, European Patent Application 0131624B1, European Patent Applications 120516, 159418B1, European Patent Applications 120516, 159418B1 and 176,112 to Schilperoot, U.S. Pat. Nos. 5,149,645, 5,469,976, 5,464,763 and 4,940,838 and 4,693,976 to Schilperoot, European Patent Applications 116718, 290799, 320500 all to MaxPlanck, European Patent Applications 604662 and 627752 to Japan Tobacco, European Patent Applications 0267159, and 0292435 and U.S. Pat. No. 5,231,019 all to Ciba Geigy, U.S. Pat. Nos. 5,463,174 and 4,762,785 both to Calgene, and U.S. Pat. Nos. 5,004,863 and 5,159,135 both to Agracetus. Other transformation technology includes whiskers technology, see U.S. Pat. Nos. 5,302,523 and 5,464,765 both to Zeneca. Electroporation technology has also been used to transform plants, see WO 87/06614 to Boyce Thompson Institute, U.S. Pat. Nos. 5,472,869 and 5,384,253 both to Dekalb, WO9209696 and WO9321335 both to PGS. All of these transformation patents and publications are incorporated by reference. In addition to numerous technologies for transforming plants, the type of tissue which is contacted with the foreign genes may vary as well. Such tissue would include but would not be limited to embryogenic tissue, callus tissue type I and II, hypocotyl, meristem, and the like. Almost all plant tissues may be transformed during dedifferentiation using appropriate techniques within the skill of an artisan.

Foreign genetic material introduced into a plant may include a selectable marker. The preference for a particular marker is at the discretion of the artisan, but any of the following selectable markers may be used along with any other gene not listed herein which could function as a selectable marker. Such selectable markers include but are not limited to aminoglycoside phosphotransferase gene of transposon Tn5 (Aph II) which encodes resistance to the antibiotics kanamycin, neomycin and G418, as well as those genes which code for resistance or tolerance to glyphosate; hygromycin; methotrexate; phosphinothricin (bar); imidazolinones, sulfonylureas and triazolopyrimidine herbicides, such as chlorosulfuron; bromoxynil, dalapon and the like.

In addition to a selectable marker, it may be desirous to use a reporter gene. In some instances a reporter gene may be used without a selectable marker. Reporter genes are genes which are typically not present or expressed in the recipient organism or tissue. The reporter gene typically encodes for a protein which provide for some phenotypic change or enzymatic property. Examples of such genes are provided in K. Weising et al. Ann. Rev. Genetics, 22, 421 (1988), which is incorporated herein by reference. Preferred reporter genes include without limitation glucuronidase (GUS) gene and GFP genes.

Once introduced into the plant tissue, the expression of the structural gene may be assayed by any means known to the art, and expression may be measured as mRNA transcribed, protein synthesized, or the amount of gene silencing that occurs (see U.S. Pat. No. 5,583,021 which is hereby incorporated by reference). Techniques are known for the in vitro culture of plant tissue, and in a number of cases, for regeneration into whole plants (EP Appln No. 88810309.0). Procedures for transferring the introduced expression complex to commercially useful cultivars are known to those skilled in the art.

Once plant cells expressing the desired level of P450 nucleic acids are obtained, plant tissues and whole plants can be regenerated therefrom using methods and techniques well-known in the art. The regenerated plants are then reproduced by conventional means and the introduced genes can be transferred to other strains and cultivars by conventional plant breeding techniques.

The following examples illustrate methods for carrying out the invention and should be understood to be illustrative of, but not limiting upon, the scope of the invention which is defined in the appended claims.

### EXAMPLES

### Example I

### Development of Plant Tissue and Ethylene Treatment.

Plant Growth

Plants were seeded in pots and grown in a greenhouse for 4 weeks. The 4-week-old seedlings were transplanted into individual pots and grown in the greenhouse for 2 months. The expanded green leaves were detached from plants to do the ethylene treatment described below. The plant material was taken from 24-48 hour post ethylene treated leaves for RNA extraction. Another subsample was taken for alkaloids analysis to confirm the concentration of nornicotine in these samples.

Tobacco Line 78379

Tobacco line 78379, a public burley line released by the University of Kentucky, was used as a source of plant material. A total of 100 plants were transplanted and tagged with a distinctive number (1-100). Fertilization and field management were conducted as recommended.

Three quarters of the 100 plants converted between 20 and 100% of the nicotine to nornicotine. One quarter of the 100 plants converted less than 5% of the nicotine to nornicotine. The range of nornicotine conversion varied greatly. For-example, plant number 87 had the least conversion (2%) while plant number 21 had 100% conversion. Plants converting less than 3% were classified as non-converters. Self-pollinated seed of plant number 87 and plant number 21, as well as crossed (21×87 and 87×21) seeds were made to study genetic and phenotype differences. Plants derived from self crossing plant number 21 were converters, and 99% of plants derived from self crossing plant 87 were non-converters. The other 1% of the plants from plant number 87 showed low conversion (5-15%). Plants from reciprocal crosses were all converters.

Tobacco Line 4407

Tobacco line 4407 was a burley line was used as a source of plant material. Uniform and representative plants totaling 100 were selected and tagged. Of the 100 plants 97 were non-converters and three were converters. Plant number 56 had the least amount of conversion (1.2%) and plant number 58 had the highest level of conversion (96%). Self-pollenated seeds and crossed seeds were generated with these two plants as described above.

Plants derived from seed that had been obtained from crossing plant number 58 with itself were segregating in about a 3:1 converter to non-converter ratio. Plants of self crossed seed of plant number 56 had 99% converters with the remaining 1% showing low conversion (5-15%). The plants from reciprocal crosses also segregated in a ratio of about 1:1.

Ethylene Treatment Procedures

One leaf from each plant was sprayed with 3 ml ethylene form Prep brand Ethephon (Rhone-Poulenc). Each sprayed leaf was hung in a curing rack equipped with humidifier and covered with plastic. Each leaf was sampled at day 5 to determine alkaloid concentration.

Alternatively, plug germinated seedlings were put into float trays in water containing 150 ppm NPK fertilizer. Seedling (4-8 weeks old) were sprayed with ethylene and cured. Ethylene treated samples were subjected directly to alkaloids analysis without further curing.

Alkaloid Analysis

Samples (0.1 g) were shaken at 150 rpm with 0.5 ml 2N NaOH, and a 5 ml extraction solution which contained quinoline as an internal standard and methyl t-butyl ether. Samples were analyzed on a HP 6890 GC equipped with a FID detector. A temperature of 250° C. was used for the detector and injector. An HP column (30 m-0.32 nm-1·m) consisting of fused silica crosslinked with 5% phenol and 95% methyl silicon was used at a temperature gradient of 110-185° C. at 10° C. per minute. The column was operated at a flow rate at 100° C. at 1.7 cm³ min⁻¹ with a split ratio of 40:1 with a 2:1 injection volume using helium as the carrier gas.

### Example 2

### RNA Isolation

For RNA extractions, middle leaves from 2 month old greenhouse grown plants were treated with ethylene as described. Samples were collected at 0 and 24 hours and used for RNA extraction. Total RNA was isolated using Rneasy Plant Mini Kit (Qiagen) following manufacturer's protocol.

100 mg of plant leaf tissue was ground with a mortar and pestle in the presence of liquid nitrogen. RNA was dissolved into 100:1 Rnase free water. Quality and quantity of total RNA was analyzed by denatured formaldehyde gel and spectrophotometer.

Total Poly (A+)RNA was isolated using Oligotex poly A RNA purification kit (Qiagen) following manufacture's protocol. About 200 ug total RNA in 250:1 maximum volume was used. Poly A+ product was analyzed by denatured formaldehyde gels and spectrophotometric analysis.

### Example 3

### Reverse Transcription-PCR

First strand cDNA was produced using SuperScript reverse transcriptase (Gibco BRL) following manufacturer's protocol. PCR was carried out with the following specification:

200 pmoles of forward primer (degenerate primers as in FIG. 1) and 100 moles reverse primer (mix of oligo d(T)+1 random base) were used in PCR reactions.

Reaction conditions were 94° C. for 2 minutes and then 40 cycles of PCR at 94° C. for 1 minute, 45° C. for 2 minutes, 72° C. for 3 minutes were performed.

Ten uL of the amplified sample were analyzed by electrophoresis using a 1% agarose gel.

### Example 4

### Generation of PCR Fragment Populations

PCR fragments from Example 3 were ligated into a pGEM-T Easy Vector (Promega) following manufacturer's instructions. Ligated product was transformed into JM109 competent cells and plated on LB media plates for blue/white selection. Colonies were selected and grown in 10 ml of LB media overnight at 37° C. Frozen stocks were generated for all selected colonies. Plasmid DNA was purified and minipreped using Wizard SV Miniprep kit (Promega). Plasmids were digested by EcoR1 and were analyzed using 1% agarose gel. The plasmids containing a 400-600 bp insert were sequenced using a ABI 3700 DNA Sequencer (Applied Biosystems). Sequences were aligned with GenBank database by BLAST search. The P450 related fragments were further analyzed.

### Example 5

### Characterization of Cloned Fragments

One step RT-PCR (Gibco Kit) was performed on the total RNAs from non-converter (58-25) and converter (58-33) lines using primers specific to the P450 fragments (FIG. 1).

### Example 6

### Preparation of Subtraction Libraries

A subtraction library was made using 58-33 (converter) as tester and 58-25 (non-converter) as driver based on the protocol provided by the manufacturer's instructions (Clontech PCR-Select cDNA Subtraction Kit). PCR fragments were ligated into pGEM plasmids. DNA was extracted by miniprep from bacterial culture grown from a single colony.

P450 clones were identified from both degenerate primer populations and the subtraction library. Nonradioactive Southern blotting was performed on most P450 clones identified. It was observed that the level of expression among different P450 clusters was very different. Further real time detection was conducted on those with high expression. The assay was also applied on the subtraction library.

### Example 7

### Identification of Appropriate Candidates

Southern blotting was conducted to identify clones differentially expressed only in converter material (vs. nonconverter material). Nonradioactive southern blotting procedures were conducted as follows.

1) Total RNA was extracted from ethylene treated converter (58-33) and nonconverter (58-25) cell leaves.

2) First step RT-PCR was conducted to biotin-tail label the single strand cDNA from converter and nonconverter total RNA (Promega, Biotinalyted oligo dT; Gibco, Superscript reverse transcriptase). These were used as a probe to hybridize with cloned DNA.

3) Plasmid DNA was digested with restriction enzyme EcoRI and run on agarose gels. 4) Gels were simultaneously dried and transferred to two membranes. One membrane was hybridized with converter probe and the other with nonconverter probe. The hybridized and washed membranes were detected by alkaline phosphatase labeling followed by NBT/BCIP colometric detection (Enzo Diagnostics, Inc.) followed by manufacture's hybridization and detection procedure with modification of stringency washes.

Comparative RT-PCR was conducted as follows.

1) Total RNA from ethylene treated converter (58-33) and nonconverter (58-25) plant leaves was extracted.

2) poly(A+) RNA from total RNA was extracted.

3) One step RT-PCR was conducted using primers specific to P450s (Gibco, one step RT-PCR system).

4) Samples were run on 1.5% agarose gels to resolve bands.

### Example 8

### Screening of cDNA P450 Candidate Clones

A cDNA library was constructed by preparing total RNA from ethylene treated leaves as follows. First, total RNA was extracted from ethylene treated leaves using a modified acid phenol and chloroform extraction protocol. The protocol was modified to use 1 g of tissue that was ground and subsequently vortexed in 5 ml of extraction buffer (100 mM Tris-HCl, pH 8.5; 200 mM NaCl; 10 mM EDTA; 0.5% SDS) to which 5 ml phenol (pH 5.5) and 5 ml chloroform was added. The extracted sample was centrifuged and the supernatant was saved. This extraction step was repeated 2-3 more times until the supernatant appeared clear. Approximately 5 ml of chloroform was added to remove trace amounts of phenol. RNA was precipitated from the combined supernatant fractions by adding a 3-fold volume of ETOH and 1/10 volume of 3M NaOAc (pH 5.2) and storing at −20° C. for 1 hour. After transfering to Corex glass container it was centrifuged at 9,000 RPM for 45 minutes at 4° C. The pellet was washed with 70% ethanol and spun for 5 minutes at 9,000 RPM at 4° C. After drying the pellet, the pelleted RNA was dissolved in 0.5 ml RNase free water. The pelleted RNA was dissolved in 0.5 ml RNase free water. The quality and quantity of total RNA was analyzed by denatured formaldehyde gel and spectrophotometer, respectively.

The resultant total RNA was isolated for poly A+ RNA using an Oligo(dT) cellulose protocol (Invitrogen) and Microcentrifuge spin columns (Invitrogene) by the following protocol. Approximately 20 mg of total RNA was subjected to twice purification to obtain high quality poly A+ RNA. Poly A+ RNA product was analyzed by performing denatured formaldehyde gel and subsequent RTPCR of control full-length genes to ensure high quality of mRNA. In addition, Northern analysis was performed on the poly A+ RNA from ethylene treated non-converter leaves, zero hour ethylene treated converter leaves and ethylene treated converter leaves using a full-length P450 gene as probe. The method was based on the protocol provided by the manufacturer's instructions (KPL RNADetector Northern Blotting Kit) using 1.8 ug of polyA+ RNA for each sample. RNA containing gels were transferred overnight using 20×SSC as a transfer buffer.

Next, poly A+ RNA was used as template to produce a cDNA library employing cDNA synthesis kit, ZAP-cDNA synthesis kit, and ZAP-cDNA Gigapack III Gold cloning kit (Stratagene). The method involved following the manufacture's protocol as specified. Approximately 8 ug of poly A+ RNA was used to a construct cDNA library. Analysis of the primary library revealed about 2.5×10⁶−1×10⁷ pfu. A quality background test of the library was completed by complementation using IPTG and X-gal, where recombinant plaques were expressed at more than 100-fold above the background reaction.

A more quantitative analysis of the library by random PCR showed that average size of insert cDNA was approximately 1.2 kb. The method used a two-step PCR method as followed. For the first step, reverse primers were designed based on the preliminary sequence information obtained from P450 fragments. The designed reverse primers and T3 (forward) primers were used amplify corresponding genes from the cDNA library. PCR reactions were subjected to agarose electrophoresis and the corresponding bands of high molecular weight were excised, purified, cloned and sequenced.

Numerous modifications and variations in practice of the invention are expected to occur to those skilled in the art upon consideration of the foregoing detailed description of the invention. Consequently, such modifications and variations are intended to be included within the scope of the following claims.

What is claimed is:

1. An isolated nucleic acid molecule, wherein said nucleic acid molecule comprises a nucleic acid sequence selected from the group consisting of SEQ. ID. 13, 15, 17,19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

2. The isolated nucleic acid molecule of claim 1, wherein said nucleic acid molecule comprises a fragment of a cytochrome P450 gene.

3. An isolated nucleic acid molecule, wherein said nucleic acid molecule has at least 80% identity to the nucleic acid molecule of claim 1.

4. An isolated nucleic acid molecule, wherein said nucleic acid molecule has at least 90% identity to the nucleic acid molecule of claim 1.

5. An isolated nucleic acid molecule, wherein said nucleic acid molecule has at least 95% identity to the nucleic acid molecule of claim 1.

6. An isolated nucleic acid molecule, wherein said nucleic acid molecule has at least 98% identity to the nucleic acid molecule of claim 1.

7. A transgenic plant, wherein said transgenic plant comprises the nucleic acid molecule of claim 1, 2, 3, 4, 5, or 6.

8. The transgenic plant of claim 7, wherein said plant is a tobacco plant.

9. A method of producing a transgenic plant, said method comprising the steps of:
(i) operably linking the nucleic acid molecule of claims 1, 2, 3, 4, 5, or 6, with a promoter functional in said plant to create a plant transformation vector; and
(ii) transforming said plant with said,plant transformation vector of step (i);
(iii) selecting a plant cell transformed with said transformation vector; and
(iv) regenerating a plant from said plant cell of step (iii).

10. The method of claim 9, wherein said nucleic acid molecule is in an antisense orientation.

11. The method of claim 9, wherein said nucleic acid molecule is in a sense orientation.

12. The method of claim 9 wherein said nucleic acid is in a RNA interference orientation.

13. The method of claim 12, wherein said nucleic acid molecule is expressed as a double stranded RNA molecule.

14. The method of claim 12, wherein said double stranded RNA molecule is about 15 to 25 nucleotide in length.

15. The method of claim 9, wherein said plant is a tobacco plant.

16. A method of selecting a plant containing a nucleic acid molecule, wherein said plant is analyzed for the presence of nucleic acid sequence, wherein said nucleic sequence acid selected from the group consisting of SEQ. ID 13, 15, 17, 19, 21, 23, 25, 27, 29, 31, 33, 35, 37, 39, 41, 43, 45, 47, 49, 50, 51, 53, 55, 57, 59, 61, 63, 65, 67, 69, 71, 73, 75, 77, 79, 81, 83, 85, 87, 89, 91, 92, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

17. The method of selecting a plant of claim 16, wherein said plant is analyzed by DNA hybridization.

18. The method of selecting a plant of claim 16, wherein said plant is analyzed by PCR detection.

19. The method of claim 16, wherein said DNA hybridization comprises a nucleic acid probe, said nucleic acid probe is selected from a group consisting of SEQ. ID. 13, 15, 17,19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91 93, 95, 97, 99, 101, 103, 105, 107, 109, 111, 113, 115, 117, 119, 121, 123, 125, 127, 129, 131, 133, 135, 137, 139, 141, 143, 145, 147, 149, or 151.

20. The method of selecting a plant of claim 16, wherein said plant is a transgenic plant.

21. The method of selecting a plant of claim 16, wherein said plant is selected from a mutagenesis population.

22. The method of selecting a plant of claim 16, wherein said plant is selected from a breeding population.


SEQUENCE LISTING (152 sequences)

SEQ ID NO: 1 - DNA, 4 bp, Nicotiana
ktry

SEQ ID NO: 2 - DNA, 4 bp, Nicotiana
ktrr

SEQ ID NO: 3 - DNA, 1 bp, Nicotiana
r

SEQ ID NO: 4 - DNA, 1 bp, Nicotiana
r

SEQ ID NO: 5 - DNA, 4 bp, Nicotiana
grrc

SEQ ID NO: 6 - DNA, 4 bp, Nicotiana
ggrr

SEQ ID NO: 7 - DNA, 17 bp, Nicotiana
aargaracyttmgytta

SEQ ID NO: 8 - DNA, 16 bp, Nicotiana
aargaracytmgytmg

SEQ ID NO: 9 - DNA, 13 bp, Nicotiana
ttyccgarmgtty

SEQ ID NO: 10 - DNA, 14 bp, Nicotiana
raackytgcggraa

SEQ ID NO: 11 - DNA, 13 bp, Nicotiana
ggmgmgtgyccgs

SEQ ID NO: 12 - DNA, 13 bp, Nicotiana
ckckccccraagg

SEQ ID NO: 13 - DNA, 183 bp, Nicotiana
gggcggcgggggtgtccggggatgacttatgcattacaagttacaaaactccaaatgacgagcccctggatatgaaggaaggtgcaggattaactatacgtaaagtaaatcctgtagaagtgacaattacggctcgcctggcacctgagctttattaaaaccttagatgttttatcttgatta

SEQ ID NO: 14 - protein, 65 aa, Nicotiana
GRRGCPGMTYALQVEHLTIAHLIQGFNYKTPNDEPLDMKEGAGLTIRKVNPVEVTITARLAPELY

SEQ ID NO: 15 - DNA, 286 bp, Nicotiana
gggaggcggggggtgtccggggatgacttatgcattacaagtggaacacctaacaatagcacatttgatccagggtttcaattacaaaactccaaatgacgagcccttggatataaaaggtgcaggattaactatacgtaaagtaaatcctgtagaagtgacaattacggctcgcctggcacctgagctttattaaaaccttagatgttttatcttgattgtactaatatatatatgcagaaaaaattgaaatgaaatgtgatcgaaattgtgtacggttggataa

SEQ ID NO: 16 - protein, 65 aa, Nicotiana; identical to SEQ ID NO: 14

SEQ ID NO: 17 - DNA, 203 bp, Nicotiana
gggcggcgggggtgcccggggatgacttatgcattacaagtggaacacctaacaatagcacatttgatccagggtttcaattacaaaactccaaatgacgagcccttggatatgaaggaaggttcaggattaaccatacgtaaagtaaatcctgtagaagtgacaactacggctcgcctggcacctgagctttattaaaacca

SEQ ID NO: 18 - protein, 64 aa, Nicotiana
GRRGCPGMTYALQVEHLTIAHLIQGENYKTPNDEPLDMKEGAGLTIRKVNPVEVTTARLAPELY

SEQ ID NO: 19 - DNA, 371 bp, Nicotiana
gggcggcgggggtgtccggggataaattttgcgactttagtgacacatctgacttttggtcgcttgcttcaaggttttgattttagtacgccatcaaacacgccaatagacatgacagaaggcgtaggcgttactttgcctaaggtaaatcaagtggaagttctaattagccctcgtttaccttctaagctttatgtattctgaaagtgcaaatcatcactcgtggcttgagtaattagttatactttaatatgtttctcgtgtaaattttatggggccgtatatggtcacttgtagtggttgtgcataaaatgaagttgtgaaatatataaacttcatataagtgccagtcttatttagtttcttgtcta

SEQ ID NO: 20 - protein, 67 aa, Nicotiana
GRRGCPGINFATLVTHLTFGRLLQGFDFSTPSNTPIDMTEGVGVTLPKVNQVEVLISPRLPSKLYVF

SEQ ID NO: 21 - DNA, 280 bp, Nicotiana
gggaggcgggggtgtccggggatgacttatgcattgcaagtggaacacctaacaatggcacatttaatccagggtttcaattacaaaactccaaatgacgaggccttggatatgaaggaaggtgcaggcataacaatacgtaaggtaaatccagtggaattgataataacgcctcgcttggcacctgagctttactaaaacctaagatctttcatcttggttgatcattgtttaatacccctagatgggtattcatttaccttttttcaattaattgcat

SEQ ID NO: 22 - protein, 64 aa, Nicotiana
GRRGCPGMTYALQVEHLTMAHLIQGFMYHTPNBEALMKEGAGITIPKVNPVELIITPRLAPELY

SEQ ID NO: 23 - DNA, 211 bp, Nicotiana
gggcggcgggggtgtccgggaatgctttggagtgcgagtatagtgcgcgtcagctacctaacttgtatttatagattccaagtatatgctgggtctgtgttcagagtagcatgaacaggcctttcctgtttgttgaatttaccccatatgtttattgcagcaggaacttgagttgagacattagagattgctggtatatatttttaagagc

SEQ ID NO: 24 - protein, 37 aa, Nicotiana
GRRGCPGMLWSASIVRVSYLTCIYRFQVYAGSVFRVA

SEQ ID NO: 25 - DNA, 376 bp, Nicotiana
gggaggcgggggtgtccggggatgacttatgcattgcaagtggaacacttaacaatggcacatttgatccaaggtttcaattacagaactccaaatgacgagcccttggatatgaaggaaggtgcaggcataactatacgtaaggtaatcctgtggaactgataatagcgcccctggcacctgagctttattaaaacctaagatctttcatcttggttgatcattctataatactcctaaatggatattcatttaccttttatcaattaattgtcagtacgagtttttctaatttggtacatttgtaataataagtaaagaataattgtgctaatatataaaggtttgtagaagataattgactggttgtaccaca

SEQ ID NO: 26 - protein, 64 aa, Nicotiana
GRRGCPGMTYALQVEHLTMAHLIQGFNYRTPNDEPLDMKEGAGITIRKVNPVELIIAPLAPELY

SEQ ID NO: 27 - DNA, 292 bp, Nicotiana
gggcggcgggggtgtccggggatgacttatgcattgcaagtggaacacctaacaatggcacatttgatccagggtttcaattacagaactccaactgatgagcccttggatatgaaagaaggtgcaggcataactatacgtaaggtaaatcctgtgaaagtgataattacgcctcgcttggcacctgagctttattaaaatctaagatgtttcatcttggttgatcattgtttaatactcctagatgggtattcatctaccttttttcaaaaaaaaaaaaaaaaaaaaaaaa

SEQ ID NO: 28 - protein, 65 aa, Nicotiana
GRRGCPGMTYALQVEHLTMAHLIQGFNYRTPTDEPLDMKEGAGITIRKVNPVKVIITPRLAPELY

SEQ ID NO: 29 - DNA, 368 bp, Nicotiana
gggcggcgggggtgtccggggatgacttatgcattgcaagtggaacacttaacaatggcacatttgatccaaggtttcaattacagaactccaaatgacgagcccttggatatgaaggaaggtgcaggcataactatacgtaaggtaaatcctgcggaactgataatagcgcctcgcctggcacctgagctttattaaaacctaagatctttcatcttggttgatcattgtataatactcctaaatggatattcatttaccttttatcaattaattgtcagtacgagtttttctaatttggtacatttgtaataataagtaaagaataattgtgctaatatataaaggtttgtagaagataattgact

SEQ ID NO: 30 - protein, 65 aa, Nicotiana
GRRGCPGMTYALQVEHLTMAHLIQGENYRTPNDEPLDMKEGAGITIRKVNPAELIIAPRLAPELY

SEQ ID NO: 31 - DNA, 257 bp, Nicotiana
gggcggagggggtgtccggggataggttttgcgactttagtgacacatctgacttttggtcgcttgcttcaaggttttgattttagtaagccatcaaacacgccaattgacatgacagaaggcgtaggcgttactttgcctaaggttaatcaagttgaagttctaattacccctcgtttaccttctaagctttatttattttgaaagtgcaaatcatcaatcatgggttgggtaattagtgatactttaatatgtta

SEQ ID NO: 32 - protein, 67 aa, Nicotiana
GRRGCPGIGFATLVTHLTEGRLLQGFDFSKPSNTPIDMTEGVGVTLPKVNQVEVLITPRLPSKLYLF

SEQ ID NO: 33 - DNA, 231 bp, Nicotiana
gggcggcgggggtgtccggggatgacttatgcattgcaagtggaacacttaacaatggcacatttgatccaaggtttcaattacagaactccaaatgacgagcccttggatatgaaggaaggtgcaggcataactatacgtaaggtaaatcctgtggaactgataatagcgcctcgcctggcacctgagctttattaaaaccttaagatctttcatcttggttgatcattg

SEQ ID NO: 34 - protein, 65 aa, Nicotiana
GRRGCPGMTYALQVEHLTMAHLIQGENYRIPNDEPLDMKEGAGITIRKVNPVELIIAPRLAPELY

SEQ ID NO: 35 - DNA, 227 bp, Nicotiana
gggcggcgggggtgtccggggatgacttatgcattgcaagtggaacacttaacaatggcacatttgatccaaggtttcaattacagaactccaaatgacgagcccttggatatgaaggaaggtgcaggcataactatacgtaaggtaaatcctgtggaactgataatagcgcctcgcctggcacctgagctttattaaaacctaagatctttcatcttggttgatca

SEQ ID NO: 36 - protein, 65 aa, Nicotiana
GRRGCPGMTYALQVEHLTMAHLIQGENYRTPNDEPLDMKEGAGITIRKVNPVELIIAPRLAPELY

SEQ ID NO: 37 - DNA, 288 bp, Nicotiana
gggcggcgggggtgtccggggataggttttgcgactttagtgacacatctgacttttggttcgcttgcttcaaggttttgattttagtaagccatcaaacacgccaattgacatgacaggaggcgtaggcgttactttgcctaaggttaatcaagttgaagttctaattacccctcgtttaccttctaagctttatttattttgaaagtgcaatcatcaatcatgggttgagtaattagtgatactttaatatgtttctcatgtaaatgttatggggccgtatatgga

SEQ ID NO: 38 - protein, 67 aa, Nicotiana
GRRGCPGIGFATLVTHLTFGRLLQGFDFSKPSNTPIDMTEGVGVTLPKVNQVEVLITPRLPSKLYLF

SEQ ID NO: 39 - DNA, 200 bp, Nicotiana
gggcggcgggggtgtccgggaatgctttggagtgcgagtatagtgcgcgtcagctacctaacctgtatttatagattccaagtatatgctgggtctgtgttcagagtagcatgaacaggcctttcctgtttgttgaatttacctcatatgtttattgcagcaggaacttgagttgagacaaaaaaaaaaaaaaaaaaaaa

SEQ ID NO: 40 - protein, 37 aa, Nicotiana; identical to SEQ ID NO: 24

SEQ ID NO: 41 - DNA, 249 bp, Nicotiana
gggaggcgggggtgcccgggtgcacaacttgctatcaacttggtcacatctatgttgggtcatttgttgcatcattttacatgggctccggccccgggggttaacccggaggatattgacttggaggagagccctggaacagtaacttacatgaaaaatccaatacaagctattccaactccaagagttgcctgcacacttgtatggacgtgtgccagtggatatgtaaaacattttgttctttccctt

SEQ ID NO: 42 - protein, 75 aa, Nicotiana
GRRGCPGAQLAINLVTSMLGHLLHHFTWAPAPGVNPEDIDLEESPGTVTYMKNPIQAIPTPRLPAHLYGRVPVDM

SEQ ID NO: 43 - DNA, 266 bp, Nicotiana
gggcggcgggggtgcccgggtgcacaacttgctatcaacttggtcacatctatgttgggtcatttcttcatcattttacatgggctccggccccgggggttaacccggaggatattgacttggaggagagccctggaacagtaacttacatgaaaaatccaatacaagctattccaactccaagattgcctgcacacttgtatggacgtgtgccagtggatatgtaaaacattttgttctttccctttttggttatatgatgagat

SEQ ID NO: 44 - protein, 47 aa, Nicotiana
GRRGCPGAQLAINLVTSMLGHLFIILHGLRPRGLTRRILTWRRALEQ

SEQ ID NO: 45 - DNA, 360 bp, Nicotiana
gggaggagggggtgtccggggatgacttatgcattgcaagtggaacacctaacaatggcacatttgatccagggtttcaattacagaactccaactgatgagccccttggatatgaaagaaggtgcaggcataactatacgtaaggtaaatcctgtgaaagtgataattacgcctcgcttggcacctgagctttattaaaatctaagatgtttcatcttggttgatcattgtttaatactcctagatgggtattcatctaccttttttcaattagttgtcggtacgtatttttttaatttggtaagtttgtaataataagtaaagaaggattgtgctaataaaaaaaaaaaaaaaaaaaa

SEQ ID NO: 46 - protein, 65 aa, Nicotiana; identical to SEQ ID NO: 28

SEQ ID NO: 47 - DNA, 244 bp, Nicotiana
gggcggcgggggtgtccgggaatgctttggagtgcgagtatagtgcgcgtcagctacctaacttgtatttatagattccaagtatatgctgggtctgtgttcagagtagcatgaacaggcctttcctgtttgttgaatttaccccatatgtttattgcagcaggaacttgagttgagacattagagattgctggtatatatttttaagagcttgctcgttttgtaaaaaaaaaaaaaaaaaaaa

SEQ ID NO: 48 - protein, 38 aa, Nicotiana
GRRGCPGMLWSASIVRRVSYLTCIYRFQVYAGSVFRVA

SEQ ID NO: 49 - DNA, 224 bp, Nicotiana
gggcggcgggggtgtccgggaatgctttggagtgcgagtatagtgcgcgtcagctacctaacttgtatttatagattccaagtatatgctgggtctgtgttcagagtagcatgaacaggcctttcctgtttgttgaatttaccccatatgtttattgcagcaggaacttgagttgagacattagagattgctggtatatatttttaagagcttctcgttttgta

SEQ ID NO: 50 - protein, 38 aa, Nicotiana; identical to SEQ ID NO: 48

SEQ ID NO: 51 - DNA, 340 bp, Nicotiana
gggcggcgggggtgtccgggtatgcaacttcggctttatgcattggaaatggctgtggcccatcttcttcattgttttacttgggaattgccagatggtatgaaaccaagtgagcttaaaatggatgatatttttggactcactgctccaagagctaatcgactcgtggctgtgcctactccacgtttgttgtgtcccctttattaattgaagaaaaaaggtggggcttttacttgcatcaaagagtggtgcttgtgatttttccaccttttggttaaatatacgaattattatgatatacgaattcttgggcacaaaaaaggagcatacgacatggtta

SEQ ID NO: 52 - protein, 68 aa, Nicotiana
GRRGCPGMQLGLYALEMAVAHLLHCFTWELPDGMKPSELKMDDIFGLTAPRANRLVAVPTPRLLCPLY

SEQ ID NO: 53 - DNA, 246 bp, Nicotiana
gggcggcgggggtgtccgggaatgctttggagtgcgagtatagtgcgcgtcagctacctaacttgtatttatagattccaagtatatgctgggtctgtgttcagagtagcatgaacaggcctttcctgtttgttgaatttacctcatatgtttattgcaggaggaacttgagttgagacattagagattgctggtatatatttttaagagcttgctcgttttgtaaaaaaaaaaaaaaaaaaaaaa

SEQ ID NO: 54 - protein, 37 aa, Nicotiana; identical to SEQ ID NO: 24

SEQ ID NO: 55 - DNA, 358 bp, Nicotiana
gggcggcgggggtgtccggggatgacttatgcattgcaagtggaacacctaacaatggcacatttgatccagggtttcaattacagaactccaactgatgagcccttggatatgaaagaaggtgcaggcataactatacgtaaggtaaatcctgtgaaagtgataattacgcctcgcttggcacctgagctttattaaaatctaagatgtttcatcttggttgatcattgtttaatactcctagatgggtattcatctaccttttttcaattagttgtcggtacgtatttttttaatttggtaagtttgtaataataagtaaagaaggattgtgctaataaaaaaaaaaaaaaaaaaa

SEQ ID NO: 56 - protein, 65 aa, Nicotiana; identical to SEQ ID NO: 28

SEQ ID NO: 57 - DNA, 290 bp, Nicotiana
gggcggcgggggtgcccgggtgcacaacttgctatcaacttggtcacacttatgttgggtcatttgttgcatcattttacgtgggctccgcccccgggggttaacccggagaatattgacttggaggagagccctggaacagtaacttacatgaaaaatccaatacaagctattcctactccaagattgcctgcacacttgtatggacgtgtgccagtggatatgtaaaacattttgttcttttcctttttggcttatttttttagtattaatttcttgaacacttgatg

SEQ ID NO: 58 - protein, 75 aa, Nicotiana
GRRGCPGAQLAINLVTSMLGHLLHHFTWAPPPGVNPENIDLEESPGTVTYMKNPIQAIPTPRLPAHLYGRVPVDM

SEQ ID NO: 59 - DNA, 347 bp, Nicotiana
gggcggagggggtgtccgggagaaggattggctgttcgaatggttgccttgtcattgggatgtattattcaatgttttgattggcaacgaatcggcgaagaattggttgatatgactgaaggaactggacttactttgcctaaagctcaacctttggtggccaagtgtagcccacgacctaaaatggctaatcttctctctcagatttgaacataattggtttctaccaacatccccacaactagaattttattattggtaacctatatcaatgtaatcaattttaaaccatattatatctcaatgtattccttttttatttgtttaaaaaaaaaaaaaaaaaaaaa

SEQ ID NO: 60 - protein, 69 aa, Nicotiana
GRRGCPGEGLAVRMVALSLGCIIQCFDWQRIGEELVDMTEGTGLTLPKAQPLVAKCSPRPKMANLLSQI

SEQ ID NO: 61 - DNA, 314 bp, Nicotiana
gggaggcgggggtgtccggggatgacttatgcattgcaagtggaacacctaacaatggcacatttaatccaggtttcaattacaaaactccaaatgacgaggccttggatatgaaggaaggtgcaggcataactatacgtaaggtaaatcctgtggaactgataatagcgcctcgcctggcacctgagctttattaaaacctaagatctttcatcttggttgatcattgtataatactcctaaatggatattcatttaccttttatcaattaattgtcagtacgagtttttctaaaaaaaaaaaaaaaaaaaaa

SEQ ID NO: 62 - protein, 65 aa, Nicotiana
GRRGCPGMTYALQVEHLTMAHLIQGFNYKTPMDEALDMKEGAGITIRKVNPVELIIAPRLAPELY

SEQ ID NO: 63 - DNA, 400 bp, Nicotiana
gggcggcgggggtgtccggggatgacttatgcattgcaagtggaacacctaacaatggcacatttaatccagggtttcaattacaaaactccaaatgacgaggccttggatatgaaggaaggtgcaggcataacaatacgtaaggtaaatccagtggaattgataataacgcctcgcttggcacctgagctttactaaaacctaagatctttcatcttggttgatcattgtttaatactcctagatgggtattcatttaccttttttcaattaattgcatgtacgagcttttttaatttggtatatttgtaacaataagtaaagaatgattgtgctaatatataaagatttgcagaagataattgactgattgtaccacaatttcaaaaaaaaaaaaaaa

SEQ ID NO: 64 - protein, 65 aa, Nicotiana
GRRGCPGMTYALQVEHLTMAHLIQGFNYKTPNDEALDMKEGAGITIRKVNPVELIITPRLAPELY

SEQ ID NO: 65 - DNA, 395 bp, Nicotiana
gggaggagggggtgtccggggatttcgtttggtttagctaatgcttatttgccattggctcaattactttatcactttgattgggaactccccactggaatcaaaccaagcgacttggacttgactgagttggttggagtaactgccgctagaaaaagtgacctttacttggttgcgactccttatcaacctcctcaaaactgatttaatgactttagtgttttcaattttttatttcctagtaaaccccactgttgtcctatctttcttttggtgtttttctgattttatctactctaatacatgtatcttttaccatataggaatgtatcgtgttgtcaaataacattttctgtttatctcaaattttggaataaaaaaaaaaaaaaaaaaaa

SEQ ID NO: 66 - protein, 67 aa, Nicotiana
GRRGCPGISFGLANAYLPLAQLLYHFDWELPTGIKPSDLDLTELVGVTAARKSDLYLVATPYQPPQN

SEQ ID NO: 67 - DNA, 288 bp, Nicotiana
gggcggcgggggtgtccggggataggttttgcgactttagtgacacatctgacttttggtcgcttgcttcaaggttttgattttagtaagccatcaaacacgccaattgacatgacagaaggcgtaggcgttactttgcctaaggttaatcaagttgaagttctaattacccctcgtttaccttctaagctttatttattttgaaagtgcaaatcatcaatcatgggttgagtaattagtgatactttaatatgtttctcatgtaaatgttatggggccgtatatgga

SEQ ID NO: 68 - protein, 68 aa, Nicotiana
GRRRGCPGIGFATLVTHLTFGRLLQGFDFSKPSNTPIDMTEGVGVTLPKVNQVEVLITPRLPSKLYLF

SEQ ID NO: 69 - DNA, 321 bp, Nicotiana
gggcggagggggtgtccgggagaaggttggctgttcgaatggttgccttgtcattgggatgtattgttcaatgttttgattggcaacgaatcggcgaagaattggttgatatgactgaaggaactggacttactttgcctaaagctcaacctttggtggccaagtgtagcccacgacctaaaatggctaatcttctctctcagatttgaacataattggtttctaccaacatccccacaactagaattttattattggtaacctatatcaatgtaatcaattttaaaccatattatatctcaatgtattccttttttattt

SEQ ID NO: 70 - protein, 69 aa, Nicotiana
GRRGCPGEGLAVRMVALSLGCIIQCFDWQRTGEELVDMTEGTGLTLPKAQPLVAKCSPRPKMANLLSQI

SEQ ID NO: 71 - DNA, 244 bp, Nicotiana
gggcggcgggggtgtccgggaatgctttggagtgcgagtatagtgcgcgtcagctacctaacttgtatttatagattccaagtatatgctgggtctgtgtccagagtagcatgaacaggcctttcctgtttgttgaatttacctcatatgtttattgcagcaggaacttgagttgagacattagagattgctggtatatatttttaagagcttgctcgttttgtaaaaaaaaaaaaaaaaaaaa

SEQ ID NO: 72 - protein, 37 aa, Nicotiana
GRRGCPGMLWSASIVRVSYLTCIYRFQVYAGSVSRVA

SEQ ID NO: 73 - DNA, 419 bp, Nicotiana
gggaggagggggtgtccgggctatagccttggacttaaggttatccgagtaacattagccaacatgttgcatggattcaactggaaattacctgaaggtatgaagccagaagatataagtgtggaagaacattatgggctcactacacatcctaagtttcctgttcctgtgatcttggaatctagactttcttcagatctctattcccccatcacttaatcctaagtggcttcctattatagcatcatatcaatatccctctaataaatagaggatagttgtcataggaaggaacctatgcctaaagttttggattactactaaaactgaacaacttttaggtttttgtctattctgttccctaaacaaaagaagacatctatcaataaaatagctcttatatctaaaaaaaaaaaaaa

SEQ ID NO: 74 - protein, 72 aa, Nicotiana
GRRGCPGYSLGLKVIRVTLANMLHGFNWKLPEGMKPEDISVEEHYGLTTHPKFPVPVILESRLSSDLYSPIT

SEQ ID NO: 75 - DNA, 364 bp, Nicotiana
gggaggagggggtgtccgggaatgctatttggtttagctaatgttgggacaagctttagctcagttactttatcacttcgattggaaactccctaatggacaaagtcatgagaatttcgacatgactgagtcacctggaatttctgctacaagaaaggatgatcttgttttgattgccactccttatgattcttattaagcagtagcagaaataaaaagccggggcaaacagaaaaaagtattgctgcttctaggtattttctattggataaatttcaaaattcatccacaatatttagtgtttgctagagttggtcagttttccagtctatatcatctatatgtactcaataattgtatggga

SEQ ID NO: 76 - protein, 65 aa, Nicotiana
GRRGCPGMLFGLANVGQPLAQLLYHFDWKLPNGQSHENFDMTESPGISATRKDDLVLIATPYDSY

SEQ ID NO: 77 - DNA, 445 bp, Nicotiana
gggcggcgggggtgcccgggttatagcttggggctcaaggtgattcaagctagcttagctaatcttctacatggatttaactggtcattgcctgataatatgactcctgaggacctcaacatggatgagatttttgggctctctacacctaaaaaatttccacttgctactgtgattgagccaagactttcaccaaaactttactctgtttgattcagcagttctatggttccgtcaagatagactttgttacgtttgaacctgtgctctaaatcttttgtaatggtatcgtctactcatccaacttaaatcttgtatctttttctttgcttgaaagtggttttaatagtgaacacacaagtatttatgtatgtatgttataatgcagttatattttcagaaataataacattacagtgttgtgttaaaaaaaaaaaaaaaaaaa

SEQ ID NO: 78 - protein, 70 aa, Nicotiana
GRRGCPGYSLGLKVIQASLANLLHGFNWSLPDNMTPEDLNMDEIFGLSTPKKFPLATVIEPRLSPKLYSV

SEQ ID NO: 79 - DNA, 434 bp, Nicotiana
gggcggcgggggtgtccgggaatgctttggagtgcgagtatagtgcgcgtcagctacctaacttgtatttatagattccaagtatatgctgggtctgtgttcagagtagcatgaacaggcctttcctgtttgttgaatttacctcatatgtttattgcagcaggaacttgagttgagacattagagattgctggtatatatttttaagagcttgctcgttttgtacatgttccttttagagtaggaccttaccgttgatttcccttcagcagattttagacgaaacttttaatttgcgattttatgttcaccctatatgggaaagtatggcacgttgtcctcacgggctatattgaagagaagtggtaactatgtattagcaagatctatatctaatttaccgttaatttcttcaaaaaaaaaaaaaaaaaaaa

SEQ ID NO: 80 - protein, 37 aa, Nicotiana; identical to SEQ ID NO: 24

SEQ ID NO: 81 - DNA, 451 bp, Nicotiana
gggaggcgggggtgcccgggttatagcttggggctcaaggtgattcaagctagcttagctaatcttctacatggattaactggtcattgcctgataatatgactcctgaggacctcaacatggatgagatttttgggctctctacacctaaaaaatttccacttgctactgtgattgagccaagactttcaccaaaactttactctgtttgattcaggagttctatggttccgtcaagatagactttgttacgtttgaacctgtgctctaaatcctttgtaatggtatcgtctacttatccaacttaaatcttgtatctttttctttgcttgaaagtggttttaatagtgaacacacaagtatttatgtatgtatgttataatgcagttatattttcagaaataataacattacagtgttgtgtttgtaaaaaaaaaaaaaaaaaaaaaaa

SEQ ID NO: 82 - protein, 70 aa, Nicotiana; identical to SEQ ID NO: 78

SEQ ID NO: 83 - DNA, 449 bp, Nicotiana
gggaggcgggggtgcccgggttatagcttggggctcaaggtgattcaagctagcttagctaatcttctacatggattaactggtcattgcctgataatatgactcctgaggacctcaacatggatgagatttttgggctctctacacctaaaaaatttccacttgctactgtgattgagccaagactttcaccaaaactttactctgtttgattcaggagttctatggttccgtcaagatagactttgttacgtttgaacctgtgctctaaatcctttgtaatggtatcgtctacttatccaacttaaatcttgtatctttttctttgcttgaaagtggttttaatagtgaacacacaagtatttatgtatgtatgttataatgcagttatattttcagaaataataacattacagtgttgtgtttgtaaaaaaaaaaaaaaaaaaaaa

SEQ ID NO: 84 - protein, 70 aa, Nicotiana; identical to SEQ ID NO: 78

SEQ ID NO: 85 - DNA, 451 bp, Nicotiana
gggaggcgggggtgcccgggttatagcttggggctcaaggtgattcaagctagcttagctaatcttctacatggattaactggtcattgcctgataatatgactcctgaggacctcaacatggatgagatttttgggctctctacacctaaaaaatttccacttgctactgtgattgagccaagactttcaccaaaactttactctgtttgattcaggagttctatggttccgtcaagatagactttgttacgtttgaacctgtgctctaaatcctttgtaatggtatcgtctacttatccaacttaaatcttgtatctttttctttgcttgaaagtggttttaatagtgaacacacaagtatttatgtatgtatgttataatgcagttatattttcagaaataataacattacagtgttgtgtttgttctaaaaaaaaaaaaaaaaaaaa

SEQ ID NO: 86 - protein, 70 aa, Nicotiana; identical to SEQ ID NO: 78

SEQ ID NO: 87 - DNA, 344 bp, Nicotiana
gggaggagggggtgtccgggaattatacttgcattgccaattcttggcatcagtttgggacgtttggttcagaactttgagctgttgcctcctccaggccagtcgaagctcgacaccacagagaaaggtggacagttcagtctccacattttgaagcattccaccattgtgttgaaaccaaggtctttctgaactttgtgatcttattaattaaggggttctgaagaaatttgatagtgttggatatttttatttgattaaagacgttgaagtttgacagagaacattcttctttttatgttatatatagtcttgttggactaaaaaaaaaaaaaaaaaaaaaa

SEQ ID NO: 88 - protein, 63 aa, Nicotiana
GRRGCPGIILALPILGITLERLVQNFELLPPPGQSKLDTTEKGGQFSLHILKHSTIVLKPRSF

SEQ ID NO: 89 - DNA, 234 bp, Nicotiana
gggcggcgggggtgtccgggatacagtcttgggattcgtataattagggcaactttagctaacttgttgcatggattcaactggagattgcctaatggtatgagtccagaagacattagcatggaagagatttatgggctaattacacaccccaaagtcgcacttgacgtgatgatggagcctcgacttcccaaccatctttacaaatagtggataattaaaaccattaaaatc

SEQ ID NO: 90 - protein, 69 aa, Nicotiana
GRRGCPGYSLGIRIIRATLANLLHGFNWRLPNGMSPEDISMEEIYGLITHPKVALDVMMEPRLPNHLYK

SEQ ID NO: 91 - DNA, 137 bp, Nicotiana
gggcggcgggggtgtccggccctgtgctttccatgtttaatctctagttatatactggctttgaatgtgaatctgtatcataatttcttgcaaatttctccttccatttcttattaaaaaaaaaaaaaaaaaaaaaa

SEQ ID NO: 92 - protein, 38 aa, Nicotiana
GRRGCPALCFPCLISSYILALNVNLYHNFLQISPSISY

SEQ ID NO: 93 - DNA, 364 bp, Nicotiana
cacgaaaagtccattgatgttaaaggacatgattttgagcttttgccatttggagctgggagaaggatgtgcccgggttataacttggggcttaaggtgattcaagctagcttagctaatcttatacatggatttaactggtcattgcctgataatatgactcctgaggacctcgacatggatgagatttttgggctctccacacctaaaaagtttccacttgctactgtgattgagccaagactttcacctaaactttactctgtttgattcagcacttctgtggttccatcaagatagactctttgttatgtttgaactcgtgctttatatcttttgtaatggtatcgtctaatatcgaatt

SEQ ID NO: 94 - protein, 89 aa, Nicotiana
HEKSIDVKGHDFELLPFGAGRRMCPGYNLGLKVIQASLANLIHGFNWSLPDNMTPEDLDMDEIFGLSTPKKFPLATVIEPRLSPKLYSV

SEQ ID NO: 95 - DNA, 335 bp, Nicotiana
catgaaaagtccatagatgttaaaggacatgattatgagcttttgccatttggagcggggagaagaatgtgcccgggttatagcttggggctcaaggtgattcaagctagcttagctaatcttctacatggatttaactggtcattgcctgataatatgactcctgaggacctcaacatggatgagatttttgggctctctacacctaaaaaatttccacttgctaccgtgattgagccaagactttcaccaaaactttactctgtttgattcagcagttctatggttccgtcaagatagactttgttacgtttgaacctgtgctctaaatcttt

SEQ ID NO: 96 - protein, 89 aa, Nicotiana
HEKSIDVKGHDYELLPFGAGRRMCPGYSLGLKVIQASLANLLHGFNWSLPDNMTPEDLNMDEIFGLSTPKKFPLATVIEPRLSPKLYSV

SEQ ID NO: 97 - DNA, 473 bp, Nicotiana
agtgatggaatatccaaagcaacaaaaggaaaacttgtgttttttccatttagttggggtccaagaatatgtattgggcaaaattttgctatgttagaggctaaaatggcaatggctatgattctgaaaacctatgcatttgaactctctccatcttatgctcatgctcctcatccactactacttcaacctcaatatggtgctcaattaattttgtacaagttgtagatatggtcaatttggaacttgttatggaacttttatcatcgtaatcaaccatattgagggaacatggtttgaggttaaatcctcgtgtgtgtgtcgctggtcgttgttattaccctctctactcttcggggtagggatagtgtctgcgtacatattaccctccccagaccccacttgtgggattatactgggtggttgttattgttgttgttgtactctctcaggttgtttcttgttgaccagcc

SEQ ID NO: 98 - protein, 75 aa, Nicotiana
SDGISKATKGKLVFFPFSWGPRICIGQNFAMLEAKMAMAMILKTYAFELSPSYAHAPHPLLLQPQYGAQLILYKL

SEQ ID NO: 99 - DNA, 367 bp, Nicotiana
catggaaagtccatagatgttaaaggacatgattatgagcttttgccatttggagcggggagaagaatgtgcccgggttatagcttggggctcaaggtgattcaagctagcttagctaatcttctacatggatttaactggtcattgcctgataatatgactcctgaggacctcaacatggatgagatttttgggctctctacacctaaaaaatttccacttgctactgtgattgagccaagactttcaccaaaactttactctgtttgattcagcagttctatggttccgtcaagatagactttgttacgtttgaacctgtgctctaaatcttttgtaatggtatcgtctacttatccaacttaaa

SEQ ID NO: 100 - protein, 89 aa, Nicotiana
HGKSIDVKGHDYELLPFGAGRRMCPGYSLGLKVIQASLANLLHGFNWSLPDNMTPEDLNMDEIFGLSTPKKFPLATVIEPRLSPKLYSV

SEQ ID NO: 101 - DNA, 304 bp, Nicotiana
gctgagggaattgcaacagcaacaaagaacagactttgtttcttgcctttcagttggggtcctcgtatttgcattggtaataattttgcaatgttggaaactaagattgccttagcaatgatcctacagcgttttgctttcgagctttctccatcttacgctcatgcacctacttatgtcgtcactcttcgacctcagtgtggtgctcacttaatcttgcaaaaattataggtccttaatctggatttcccattattgagtagtgcctaataaatcttctctatcactatttttccatctttca

SEQ ID NO: 102 - protein, 76 aa, Nicotiana
AEGIATATKNRLCFLPFSWGPRICIGNNFAMLETKIALAMILQRFAFELSPSYAHAPTYVVTLRPQCGAHLILQKL

SEQ ID NO: 103 - DNA, 297 bp, Nicotiana
agtgaaggagttaataaagcaacaaagggtaaatttgcatattttccatttagttggggaccaagaatatgtgttggactgaattttgcaatgttagaggcaaaaatggcacttgcattgattctacaacactatgcttttgagctctctccatcttatgcacacgctcctcatacaattatcactctgcaacctcaacatggtgctcctttgattttgcgcaagctgtagcgcggatatattgattggttatctactgtaggttactaaaacatatatcatgttttttggtcgtag

SEQ ID NO: 104 - protein, 76 aa, Nicotiana
SEGVNKATKGKFAYFPFSWGPRICVGLNFAMLEAKMALALILQHYAFELSPSYAHAPHTIITLQPQHGAPLILRKL

SEQ ID NO: 105 - DNA, 368 bp, Nicotiana
aaagaaggagtgtctaaggcaacaaacggacaagtctcatttataccatttagctggggacctcgtgtttgcattggacaaaactttgcaatgatggaagcaaaaatggcagtagctatgatactacaaaaattttcctttgaactatccccttcttatacacatgctccatttgcaattgtgactattcatcctcagtatggtgctcctctgcttatgcgcagactttaaaacatatattgctgatatttaagatcagtggcgttttattctccatgtatctttctaatactaaatagttgtgtgatgcctagcgtcgcacttttcgaattttaacattgttgttttgaaatgttatcaatgtaatc

SEQ ID NO: 106 - protein, 76 aa, Nicotiana
KEGVSKATNGQVSFIPFSWGPRVCIGQNFAMMEAKMAVAMILQKFSFELSPSYTHAPFAIVTIHPQYGAPLLMRRL

SEQ ID NO: 107 - DNA, 351 bp, Nicotiana
gaaggactagaaggtgttagagatggttacaaaatgatgccttttggttctggacgaaggagttgtcctggagaaggattggctattcgaatggttgcattgtcattgggatgtattattcaatgctttgattggcaacgacttggggaaggattggttgataagactgaaggaactggacttactttgcctaaagctcaacctttagtggccaagtgtagcccacgacctataatggctaatcttctttctcagatttgaacataattggtttctaccaaacatccccaaactagaatattattattggttacatatacaatgtaatcaattttgaaccatattatatct

SEQ ID NO: 108 - protein, 86 aa, Nicotiana
EGLEGVRDGYKMMPFGSGRRSCPGEGLAIRMVALSLGCIIQCFDWQRLGEGLVDKTEGTGLTLPKAQPLVAKCSPRPIMANLLSQI

SEQ ID NO: 109 - DNA, 253 bp, Nicotiana
tctgaaggggtatcaaaagctgcaaaagagcagatgtattttccgtttggttggggtcctcggatgtgcattgggatgaactttggcatgttagaagccaagctgattttatctcaaattctgcagcgcttttggtttgagctctctccttcctacactcatgcccctctgttgactctgattatgagaccttagtatggtgctcagacaattgtccacaaactttgactagaggttttgtatgtgagtcgta

SEQ ID NO: 110 - protein, 64 aa, Nicotiana
SEGVSKAAKEQMYFPFGWGPRMCIGMNFGMLEAKLILSQILQRFWFELSPSYTHAPLLTLIMRP

SEQ ID NO: 111 - DNA, 316 bp, Nicotiana
ttgtcaagtgcaacaaagggtcaacttacatattttccatttggctggggtcctagaatatgtattggacaaaattttgccatgttagaagcaaagatggctctgtctatgatcctgcaacgcttctcttttgaactgtctccgtcttatgcacatgcccctcagtccatattaaccgttcagccacaatatggtgctccacttattttccacaagctataatttggtacttgtgaaaggtgtcttgtacaatatgttagtagagtttattcagacttagatacatgcttcaacatggttttagtgtcaagagttc

SEQ ID NO: 112 - protein, 73 aa, Nicotiana
LSSATKGQLTYFPFGWGPRICIGQNFAMLEAKMALSMILQRFSFELSPSYAHAPQSILTVQPQYGAPLIFHKL

SEQ ID NO: 113 - DNA, 268 bp, Nicotiana
agcgaaggggtggcaaaggcaacaaaggggaaaatgacatattttccatttggtgcaggaccgcgaaaatgcattgggcaaaacttcgcgattttggaagcaaaaatggctatagctatgattctacaacgcttctccttcgagctctccccatcttatacacactctccatacactgtggtcactttgaaacccaaatatggtgctcccctaataatgcacaggctgtagtcctgtgagaatatgctatccgaggaattcagttcct

SEQ ID NO: 114 - protein, 76 aa, Nicotiana
SEGVAKATKGKMTYFPFGAGPRKCIGQNFAILEAKMAIAMILQRFSFELSPSYTHSPYTVVTLKPKYGAPLIMHRL

SEQ ID NO: 115 - DNA, 363 bp, Nicotiana
agtgaaggcgtttcaaaggcaacaaagggccaaatggcgtttttcccatttggtgcaggacctcggatatgcattgggataaactttgcaatggcagaagcgaagatggctatggctatgattctgcaacgcttctcctttgagctatctccatcttacacacatgctccacagtctgtaataactatgcaaccccaatatggtgctcctcttatattgcacaaattgtaagtgtttaagacttacatgaattgccttatcggatgaatagctatgtcaagcaaacataagttgagatattttgcaactatggtctctgctttgattcgatcacaggctagattattcaattcgcgtttgttt

SEQ ID NO: 116 - protein, 87 aa, Nicotiana
SEGVSKATKGQMAFFPFGAGPRICIGINFAMAEAKMAMAMILQRFSFELSPSYTHAPQSVITMQPQYGAPLILHKLVFKTYMNCLIG

SEQ ID NO: 117 - DNA, 354 bp, Nicotiana
caatgttctgtagatatttttggtaataattttgagtttcttccctttggcgggggacggagaatttgtcctggaatgtcatttggtttagctaatctttacttaccattggctcaattactctatcactttgactggaaactcccaaccggaatcaagccaagagacttggacttgaccgaattatcgggaataactattgctagaaagggtgacctttacttaaatgctactccttatcaaccttctcgagagtaatttactattggcataaacattttaaatttccttcatcaacctcaatattgtacaataatcattcttctggtgttataggctttatcgatttccaat

SEQ ID NO: 118 - protein, 85 aa, Nicotiana
QCSVDIFGNNFEFLPFGGGRRICPGMSFGLANLYLPLAQLLYHFDWKLPTGIKPRDLDLTELSGITIARKGDLYLNATPYQPSRE

SEQ ID NO: 119 - DNA, 259 bp, Nicotiana
agtgaaggcgtttcaaaggcaacaaagggccaaatggcgtttttcccatttggtgcaggacctcggatatgcattgggataaactttgcaatgacagaagcgaagatggctatggctatgattctgcaacgcttctcctttgagctatctccatcttacacacatgctccacagtctgtaataactatgcaaccccaatatggtgctcctcttatattgcacaaattgtaagtgtttaagacttacatgaattgcctta

SEQ ID NO: 120 - protein, 76 aa, Nicotiana
SEGVSKATKGQMAFFPFGAGPRICIGINFAMTEAKMAMAMILQRFSFELSPSYTHAPQSVITMQPQYGAPLILHKL

SEQ ID NO: 121 - DNA, 292 bp, Nicotiana
aaagaaggagtgtctaaggcaacaaacggacaagtctcatttataccatttagctggggacctcgtgtttgcattggacaaaactttgcaatgatggaagcaaaaatggcagtagctatgatactacataaattttcctttgaactatccccttcttatacacatgctccatttgcaattgtgactattcatcctcagtatggtgctcctctgcttatgcgcagactttaaaacatatattgctgatatttaagatcagtggcgttttattctccatgtatctttctaatac

SEQ ID NO: 122 - protein, 76 aa, Nicotiana
KEGVSKATNGQVSFIPFSWGPRVCIGQNFAMMEAKMAVAMILHKFSFELSPSYTHAPFAIVTIHPQYGAPLLMRRL

SEQ ID NO: 123 - DNA, 237 bp, Nicotiana
agcgaaggggtggcaaaggcaacaaaggggaaaatgacatattttccatttggtgcaggaccgcgaaaatgcattgggcaaaacttcgcgattttggaagcaaaaatggctatagctatgattctacaacgcttctccttcgagctctctccatcttatacacactctccatacactgtggtcactttgaaacccaaatatggtgctcccctaataatgcacaggctgtagtcctgt

SEQ ID NO: 124 - protein, 76 aa, Nicotiana; identical to SEQ ID NO: 114

SEQ ID NO: 125 - DNA, 328 bp, Nicotiana
gaaggattggctgttcgaatggttgccttgtcattgggatgtattattcaatgttttgattggcaacgaatcggcgaagaattggttgatatgactgaaggaactggacttactttgcctaaagctcaacctttggtggccaagtgtagcccacgacctaaaatggctaatcttctctctcagatttgaacataattggtttctaccaacatccccacaactagaattttattattggtaacctatatcaatgtaatcaattttaaaccatattatatctcaatgtattccttttttatttgtttaaaaaaaaaaaaaaaaaaaaaaa

SEQ ID NO: 126 - protein, 62 aa, Nicotiana
EGLAVRMVALSLGCIIQCFDWQRIGEELVDMTEGTGLTLPKAQPLVAKCSPRPKMANLLSQI

SEQ ID NO: 127 - DNA, 266 bp, Nicotiana
atgcaacttgggctttatgcattggaaatggctgtggcccatcttcttcattgttttacttgggaattgccagatggtatgaaaccaagtgagcttaaaatggatgatatttttggactcactgctccaaaagctaatcgactcgtggctgtgcctactccacgtttgttgtgtcccctttattaattgaagaaaaaaggtggggcttttacttgcatcaaagagtggtgcttgtgatttttccaccttttggttaaatatacgaa

SEQ ID NO: 128 - protein, 61 aa, Nicotiana
MQLGLYALEMAVAHLLHCFTWELPDGMKPSELKMDDIFGLTAPKANRLVAVPTPRLLCPLY

SEQ ID NO: 129 - DNA, 213 bp, Nicotiana
ggtcagcaagttggacttcttagaacaaccattttcatcgcctcattactgtctgaatataagctgaaacctcgctcacaccagaaacaagttgaactcaccgatttaaatccagcaagttggcttcattcgataaaaggcgaactgttagtcgatgcgattcctcgaaagaaggcggcattttaaatctttaatcttggcgctgttttaaaa

SEQ ID NO: 130 - protein, 61 aa, Nicotiana
GQQVGLLRTTIFIASLLSEYKLKPRSHQKQVELTDLNPASWLHSIKGELLVDAIPRKKAAF

SEQ ID NO: 131 - DNA, 204 bp, Nicotiana
ggttataacttggggcttaaggtgattcaagctagcttagctaatcttatacatggatttaactggtcattgcctgataatatgactcctgaggacctcgacatggatgagatttttgggctctccacacctaaaaagtttccacttgctactgtgattgagccaagactttcacctaaactttactctgtttgattcagcact

SEQ ID NO: 132 - protein, 64 aa, Nicotiana
GYNLGLKVIQASLANLIHGFNWSLPDNMTPEDLDMDEIFGLSTPKKFPLATVIEPRLSPKLYSV

SEQ ID NO: 133 - DNA, 259 bp, Nicotiana
atgctatttggtttagctaatgttggacaacctttagctcagttactttatcacttcgattggaaactccctaatggacaaagtcatgagaatttcgacatgactgagtcacctggaatttctgctacaagaaaggatgatcttgttttgattgccactccttatgattcttattaagcagtagcagaaataaaaagccggggcaaacagaaaaaagtattgctgcttctaggtattttctattggataaatttcaaaa

SEQ ID NO: 134 - protein, 58 aa, Nicotiana
MLFGLANVGQPLAQLLYHFDWKLPNGQSHENFDMTESPGISATRKDDLVLIATPYDSY

SEQ ID NO: 135 - DNA, 234 bp, Nicotiana
ggaatgctttggagtgcgagtatagtgcgcgtcagcatacctaacttgtatttatagattccaagtatatgctgggtctgtgttcagagtagcatgaacaggcctttcctgtttgttgaatttacctcatatgtttattgcagcaggaacttgagttgagacattagagattgctggtatatatttttaagagcttgctcgttttgtacaaaaaaaaaaaaaaaaaaaaaaaaa

SEQ ID NO: 136 - protein, 17 aa, Nicotiana
GMLWSASIVRVSIPNLY

SEQ ID NO: 137 - DNA, 238 bp, Nicotiana
ggtattgcacttggggttgcatccatggaacttgctttgtcaaatcttctttatgcatttgattgggagttgccttatggagtgaaaaaagaagacatcgacacaaacgttaggcctggaattgccatgcacaagaaaaacgaactttgccttgtcccaaaaaattatttataaattatattgggacgtggatctcatgctagttctgtgcggtcagctaagcttattatttttggct

SEQ ID NO: 138 - protein, 57 aa, Nicotiana
GIALGVASMELALSNLLYAFDWELPYGVKKEDIDTNVRPGIAMHKKNELCLVPKNYL

SEQ ID NO: 139 - DNA, 313 bp, Nicotiana
agtgaaggagttaataaagcaacaaagggtaaatttgcatattttccatttagttggggaccaagaatatgtgttggactgaattttgcaatgttagaggcaaaaatggcacttgcattgattctacaacactatgcttttgagctctctccatcttatgcacatgctcctcatacaattatcactctgcaacctcaacatggtgctcctttgattttgcgcaagctgtagcgcggatatattgattggttatctactgtaggttactaaaacatatatcatgttttttggtcgtagaaccttctatctttct

SEQ ID NO: 140 - protein, 76 aa, Nicotiana; identical to SEQ ID NO: 104

SEQ ID NO: 141 - DNA, 358 bp, Nicotiana
acagaggagaggcaagaggaacgggtttacaagaagaattatctagcatttggagctgggccccatggatgtgtgggacagaggtatgctataaaccatttgatgctctttattgcgttgttcacggctctgattgatttcaagaggcacaaaacggacggctgtgatgatatcgcgtatattccaaccattgctccaaaggatgattgtaaagtgttcctttcacagaggtgcactcgattcccatctttttcatgaactaattgcaccttttatttaattctgatcctcaaattggtcccattggaccatggatgtaataggaccaattgcaagaatggggtccaatgtatttgtt

SEQ ID NO: 142 - protein, 85 aa, Nicotiana
TEERQEERVYKKNYLAFGAGPHGCVGQRYAINHLMLFIALFTALIDFKRHKTDGCDDIAYIPTIAPKDDCKVFLSQRCTRFPSFS

SEQ ID NO: 143 - DNA, 502 bp, Nicotiana
catgaaaagtccatagatgttaaaggacatgattatgagcttttgccatttggagcggggagaagaatgtgcccgggttatagcttggggctcaaggtgattcaagctagcttagctaatcttctacatggatttaactggtcattgcctgataatatgactcctgaggacctcaacatggatgagatttttgggctctctacacctaaaaaatttccacttgctactgtgattgagccaagactttcaccaaaactttactctgtttgattcagcagttctatggttccgtcaagatagactttgttacgtttgaacctgtgctctaaatcttttgtaatggtatcgtctacttatccaacttaaatcttgtatctttttctttgcttgaaagtggttttaatagtgaacacacaagtatttatgtatgtatgttataatgcagttatattttcagaaataataacattacagtgttgtgtttgttcaaaaaaaaaaaaaa

SEQ ID NO: 144 - protein, 89 aa, Nicotiana; identical to SEQ ID NO: 96

SEQ ID NO: 145 - DNA, 298 bp, Nicotiana
atcgctggtgatattggcttccgtggtcaccactatgagtttatcccatttggttctggaagacgatcttgtccggggatgacttatgcattgcaagtggaacacctaacaatggcacatttaatccagggtttcaattacaaaactccaaatgacgaggccttggatatgaaggaaggtgcaggcataacaatacgtaaggtaaatccagtggaattgataataacgcctcgcttggcacctgagctttactaaaacctaagatctttcatcttggttgatcattgtttaatactcc

SEQ ID NO: 146 - protein, 84 aa, Nicotiana
IAGDIGFRGHHYEFIPFGSGRRSCPGMTYALQVEHLTMAHLIQGFNYKTPNDEALDMKEGAGITIRKVNPVELIITPRLAPELY

SEQ ID NO: 147 - DNA, 474 bp, Nicotiana
atggagggatcagataaagaaggtttcgatataacaggaagtagagagatcaagatgatgccatttggcgctggtaggagaatatgcccaggctatgctttggctatgcttcatttagagtactttgtggctaatttggtttggcattttcgatgggaggctgtggagggagatgatgttgatctttcagaaaagctagaattcaccgttgtgatgaagaatccacttcgagctcgtatctgccccagagttaactctatttgaatttggtaattactagttctttctatttgcattgttccctgttgatggacttcccccatatagtactggaagttagagggagaatgattattaatgccttgctgcaatattagcttagtagttagtagtgaatataattgaaactggatatttctatcttatgtgttgtacatttggttcattgcaaaaaaaaaaaaaaaaaaaaaaaaa

SEQ ID NO: 148 - protein, 87 aa, Nicotiana
MEGSDKEGFDITGSREIKMMPFGAGRRICPGYALAMLHLEYFVANLVWHFRWEAVEGDDVDLSEKLEFTVVMKNPLRARICPRVNSI

SEQ ID NO: 149 - DNA, 280 bp, Nicotiana
gaaggtgtgcaggccgaatcatggaagctattgccatttggaatgggaaggagagcgtgcccaggttctggacttgctcaatgtgtggttggtttagctttagcaactctagtgcagtgttttgagtggaaaagggtaagcgaagaggtggttgatttgacggaaggaaaaggtctcactatgccaaaacccgagccactcatggctaggtgcgaagctcgtgacatttttcacaaagttctttcagaaatatcttaatgttttgggagtctgaattaat

SEQ ID NO: 150 - protein, 85 aa, Nicotiana
EGVQAESWKLLPFGMGRRACPGSGLAQCVVGLALATLVQCFEWKRVSEEVVDLTEGKGLTMPKPEPLMARCEARDIFHKVLSEIS

SEQ ID NO: 151 - DNA, 383 bp, Nicotiana
aatacttctgttgatcttacaggaaatcactatcagttcattcctttcggttcaggaagaagaatgtgtccaggaatgtcgtttggtttagttaacacagggcatcctttagcccagttgctctattgctttgactggaaactccctgacaaggttaatgcaaatgattttcgcactactgaaacaagtagagtttttgcagcaagcaaagatgacctctacttgattcccacaaatcacagggagcaagaatagcttaatttaatggagttcttggaagaattaaagaagaagggctatataggtgagattttttgtatggttgcaaggtttttagttcatacaataagacaatacattatattccaaaaaaaaaaaaaaaa

SEQ ID NO: 152 - protein, 84 aa, Nicotiana
NTSVDLTGNHYQFIPFGSGRRMCPGMSFGLVNTGHPLAQLLYCFDWKLPDKVNANDFRTTETSRVFAASKDDLYLIPTNHREQE